(19) United States
(12) Patent Application Publication
(10) Pub. No.: US 2020/0303063 A1
(43) Pub. Date: Sep. 24, 2020

(54) SYSTEMS AND METHODS FOR DYNAMIC AND TAILORED CARE MANAGEMENT

(71) Applicant: Health Innovators Incorporated, Cambridge, MA (US)

(72) Inventors: Prem Prakash Sharma, Cambridge, MA (US); Christine Lih Hsieh, Newton, MA (US); Stuart Martin Kirchner, Dedham, MA (US); Elizabeth Kathryn Bisio, Boston, MA (US); Douglas Neil Kurth, Somerville, MA (US); Chien-Min Lu, Cambridge, MA (US)

(73) Assignee: Health Innovators Incorporated, Cambridge, MA (US)

(21) Appl. No.: 16/825,478

(22) Filed: Mar. 20, 2020

Related U.S. Application Data
(60) Provisional application No. 62/821,940, filed on Mar. 21, 2019.

(51) Int. Cl. G16H 40/20; G06Q 10/06; G16H 10/60; G16H 40/67; G16H 20/60; G16H 70/20; G16H 80/00; H04L 29/08; G09B 5/02; G09B 19/00

(52) U.S. Cl. CPC G16H 40/20; G06Q 10/06316; G16H 10/60; G16H 40/67; G16H 20/60; A61B 5/14532; G16H 80/00; H04L 67/142; G09B 5/02; G09B 19/0092; G09B 19/003; G16H 70/20

(57) ABSTRACT

In some embodiments, a management system is configured to dynamically generate and/or adjust a step-by-step care regimen. The care regimen, or step-by-step plan, is broken down by the system into specific actions presented to a patient at specific times with tailored guidance on how to achieve the best effect of each step. The system can be configured to build tailored care plans that span contemplation phases, preparation phases, recovery phases, and long term care phases. In further embodiments, over the treatment life cycle, the system facilitates patient care compliance, planning and education, among other options. In other embodiments, the system is configured to dynamically capture and develop implicit relationship information on a patient's care journey, and augment care options for respective patients responsive to identifying implicit connections developed within the context of the patient's care journey (e.g., specified by patient provider, procedure, physician, comorbidities, etc.).

### RELATED APPLICATIONS

This application claims priority under 35 U.S.C. § 119(e) to U.S. Provisional Application Ser. No. 62/821,940 entitled “SYSTEMS AND METHODS FOR DYNAMIC AND TAILORED CARE MANAGEMENT,” filed on Mar. 21, 2019, which application is incorporated herein by reference in its entirety.

### BACKGROUND

The inventors have realized that while medical procedures, process, and treatment have continuously improved over time, engagement with the patients receiving that care has not. In conventional approaches, there is simply a failure to fully engage patients in their treatment, and even a failure to properly prepare patients prior to treatment, and provide adequate post-treatment processes and/or follow up.

### SUMMARY

According to one aspect, provided is a management system configured to dynamically generate and/or adjust a step-by-step care regimen. The care regimen, or step-by-step plan, is broken down by the system into specific actions presented to a patient at specific times with tailored guidance on how to achieve the best effect of each step. In various embodiments, the system is configured to customize displays shown to the patient on their devices. In some examples, this includes dynamically generating and adjusting a step-by-step process for preparing for a treatment (e.g., surgery) to improve and/or ensure pre-operative compliance for improved outcomes. In further examples, step-by-step processes for post-operative or post-treatment are also provided. In such examples, the system improves post-treatment compliance by breaking post-treatment regimens into individual steps, scheduled times, and directly providing instructions in the user interface on how to perform the respective steps. Not only is patient engagement and compliance improved, but the system interactions enable identification of issues faster and more accurately than conventional approaches. Step-by-steps plans and execution can also be dynamically adjusted by the system to resolve such issues. Various executions have already demonstrated improvement in patient engagement as well as pre & post treatment compliance, ultimately yielding better health outcomes.

In some further aspects, the system is configured to build tailored care plans that span contemplation phases, preparation phases, recovery phases, and long term care phases. In further embodiments, over the treatment life cycle, the system facilitates patient care compliance, planning and education, among other options. In other embodiments, the system is configured to dynamically capture and develop implicit relationship information on a patient's care journey, and augment care options for respective patients responsive to identifying implicit connections developed within the context of the patient's care journey (e.g., specified by patient provider, procedure, physician, comorbidities, etc.). In some embodiments, the system is configured to build unique data structures to capture explicit information and facilitate generation of implicit connections that augment various care plans/journeys. In one embodiment, the system implements graph based data structures to capture and store explicit care information. The graphical data structure can be analyzed to automatically identify implicit relationship information, and augment the respective care plan accordingly.

According to one aspect a care management system is provided. The system comprises at least one processor operatively connected to a memory containing instructions, which when executed cause the at least one processor to generate a user tailored care plan, comprising daily sequences of steps to accomplish the tailored care plan; display customized interfaces to an end user specifying for a respective day each of the steps in the sequence associated with the respective day; collect remote health information based on execution of the steps in the sequence; and adjust organization of the sequence of steps based on analysis of the remote health information.

According to one embodiment, the at least one processer is further configured to calculate and display a compliance score associated with performance of the steps in the sequence. According to one embodiment, the at least one processer is further configured to automatically trigger remote advice sessions in conjunction with an execution time for a respective step. According to one embodiment, the at least one processer is further configured to automatically trigger remote advice sessions in conjunction with an execution time for a respective step based on identifying a threshold compliance score. According to one embodiment, the system is further configured to display a customized interface for respective step execution, the customized interface displaying a single selection icon for presenting video instruction on step completion, and displaying a single selection icon for triggering a remote supervision session.

According to one embodiment, the system further comprises a knowledge base of education material on patient procedure, including self-care instruction. According to one embodiment, the at least one processor is further configured to filter material in the knowledge base according to a recovery phase associated with a respective user. According to one embodiment, display of the customized interfaces includes display of an education display area, and wherein the at least one processor is configured to limit content displayed to the user within the education display area based, at least in part, on the recovery phase associated with the patient. According to one embodiment, at least one processor is further configured to filter material in the knowledge base based, at least in part, on a set of care plan tasks for a given day. According to one embodiment, display of the customized interfaces includes display of an education display area, and wherein the at least one processor is configured to limit content display to the user within the education display area based, at least in part, on the set of care plan tasks for a given day.

According to one embodiment, at least one processor is further configured to filter material in the knowledge base based, at least in part, on a recovery phase associated with a respective user and a set of care plan tasks for a given day. According to one embodiment, display of the customized interfaces includes display of an education display area, and wherein the at least one processor is configured to limit content display to the user within the education display area based, at least in part, on the recovery phase associated with the patient and the set of care plan tasks for a given day. According to one embodiment, display of the customized interfaces includes display of user entered goal information in conjunction with a summary display of a set of care plan tasks selected based on a target date.

According to one embodiment, display of the customized interfaces includes display of a progress summary view, wherein the progress summary views is configured to access and display a care plan stage associated with a respective user, and display a set of prioritized task indicators. According to one embodiment, selection in the user interface of the prioritized task indicators is configured to transition the interface to a task view for completing an associated care plan task. According to one embodiment, at least some of the set of prioritized task indicators are associated with care plan tasks having designated times for completion, and wherein the interface is further configured to dynamically flash the display of at least one prioritized task indicator upon nearing the designated time for completion. According to one embodiment, the interface is configured to increase the frequency of flashing in the display until reaching the designated time. According to one embodiment, the at least one processor is configured to capture care plan tasks and define progressive targets for display to respective users over time. According to one embodiment, the at least one processor is configured to adjust progressive targets and associated displays responsive to completion information captured or submitted by respective users.

According to one embodiment, the at least one processor is configured to defined progressive targets for blood sugar readings and adjust nutrition based activities responsive to individual patient progress. According to one embodiment, display of the customized interfaces includes display of an information aggregation display, wherein the at least one processor is configured to access information associated with scheduled task and capture needed pre-requisites for display to the user. According to one embodiment, display of the customized interfaces includes integration of acknowledgement indicators associated with pre-requisites.

According to one embodiment, the at least one processor is configured to identify a scheduled task at a location remote from a patient current location, and capture navigation information, including a required time to reach the remote location, and display navigation options to the respective user. According to one embodiment, the at least one processor is configured to enable selection of procedure based modules associated with respective operations, activities, or procedures for patients. According to one embodiment, the system is configured to access recovery stage triggers associated with the procedure based modules to dynamically adapt information resources presented in the user interface to respective patients. According to one embodiment, the at least one processor is configured to develop correlations between care plan activities and an effect on patient outcome. According to one embodiment, the at least one processor is configured to update a care plan based on developed correlations. According to one embodiment, the at least one processor is configured to develop correlations between blood sugar results and patient nutrition selection and/or timing of meals, and adjust nutrition based task to improve blood sugar results. According to one embodiment, the at least one processor is configured to create associations in a graphical database based on inferred connections between care tasks, patient profile information, and/or outcome status.

According to one embodiment the system further comprises a communication module executed by the at least one processor, configured to present a communication channel connecting a patient and a care team as a group communication session. According to one embodiment, the at least one processor is configured to enforce communication according to group communication protocols (e.g., to ensure each member of the communication group receives/has access to all communication within the channel). According to one embodiment, the at least one processor is configured to capture a set of relevant systems associated with a worsening condition, based at least in part, on a patient assigned care plan, personal medical risks, and comorbidities. According to one embodiment, the at least one processor is configured to generate a symptom checker display configured to accept user reporting of any of the relevant symptoms. According to one embodiment, the at least one processor is configured to display the symptom check in conjunction with care plan tasks.

According to another aspect, a computer implemented process for executing a care management system is provided. The method comprises the generation, by at least one processor, of a user tailored care plan comprising daily sequences of steps to accomplish the tailored care plan; display, by the at least one processor, customized interfaces to an end user specifying for a respective day each of the steps in the sequence associated with the respective day; collection, by the at least one processor, remote health information based on execution of the steps in the sequence; and adjusting, by the at least one processor, organization of the sequence of steps based on analysis of the remote health information. According to one embodiment, the method further comprises calculating and displaying a compliance score associated with performance of the steps in the sequence. According to one embodiment the method further comprises automatically triggering remote advice sessions in conjunction with an execution time for a respective step.

According to one embodiment the method further comprises automatically triggering remote advice sessions in conjunction with an execution time for a respective step based on identifying a threshold compliance score. According to one embodiment the method further comprises displaying a customized interface for respective step execution, the customized interface displaying a single selection icon for presenting video instruction on step completion, and displaying a single selection icon for triggering a remote supervision session. According to one embodiment, the method further comprises a knowledge base of education material on patient procedure, including self-care instruction. According to one embodiment the method further comprises filtering material in the knowledge base according to a recovery phase associated with a respective user.

According to one embodiment, display of the customized interfaces includes display of an education display area, and wherein the method further comprises limiting content displayed to the user within the education display area based, at least in part, on the recovery phase associated with the patient. According to one embodiment the method further comprises filter material in the knowledge base based, at least in part, on a set of care plan tasks for a given day. According to one embodiment, display of the customized interfaces includes display of an education display area, and wherein the wherein the method further comprises limiting content displayed to the user within the education display area based, at least in part, on the set of care plan tasks for a given day. According to one embodiment the method further comprises filtering material in the knowledge base based, at least in part, on a recovery phase associated with a respective user and a set of care plan tasks for a given day.

According to one embodiment, displaying the customized interfaces includes display of an education display area, and wherein the method further comprises limiting content displayed to the user within the education display area based, at least in part, on the recovery phase associated with the patient and the set of care plan tasks for a given day. According to one embodiment, displaying the customized interfaces includes displaying user entered goal information in conjunction with a summary display of a set of care plan tasks selected based on a target date. According to one embodiment, displaying the customized interfaces includes displaying a progress summary view, wherein the progress summary views are configured to access and display a care plan stage associated with a respective user, and display a set of prioritized task indicators.

According to one embodiment the method further comprises transitioning the interface to a task view for completing an associated care plan task responsive to selection in the user interface of the prioritized task indicators. According to one embodiment, at least some of the set of prioritized task indicators are associated with care plan tasks having designated times for completion, and wherein the method further comprises dynamically flashing the display of at least one prioritized task indicator upon nearing the designated time for completion.

According to one embodiment, the method further comprises increasing the frequency of flashing in the display until reaching the designated time. According to one embodiment, the method further comprises capturing care plan tasks and defining progressive targets for display to respective users over time. According to one embodiment, the method further comprises adjusting progressive targets and associated displays responsive to completion information captured or submitted by respective users. According to one embodiment, the method further comprises defining progressive targets for blood sugar readings and adjusting nutrition based activities responsive to individual patient progress. According to one embodiment, displaying the customized interfaces includes displaying an information aggregation display, and wherein the method further comprises accessing information associated with scheduled task and capturing needed pre-requisites for display to the user. According to one embodiment, displaying the customized interfaces includes integrating acknowledgement indicators associated with pre-requisites. According to one embodiment, the method further comprises identifying a scheduled task at a location remote from a patient current location, capturing navigation information, including a required time to reach the remote location, and displaying navigation options to the respective user. According to one embodiment, the method further comprises enabling selection of procedure based modules associated with respective operations, activities, or procedures for patients. According to one embodiment the method further comprises accessing recovery stage triggers associated with the procedure based modules; and dynamically adapting information resources presented in the user interface to respective patients. According to one embodiment the method further comprises developing correlations between care plan activities and an effect on patient outcome. According to one embodiment, the method further comprises updating a care plan based on developed correlations.

According to one embodiment, the method further comprises developing correlations between blood sugar results and patient nutrition selection and/or timing of meals, and adjusting nutrition based task to improve blood sugar results. According to one embodiment the method further comprises creating associations in a graphical database based on inferred connections between care tasks, patient profile information, and/or outcome status. According to one embodiment the method further comprises presenting a communication channel connecting a patient and a care team as a group communication session. According to one embodiment, the method further comprises enforcing communication according to group communication protocols (e.g., to ensure each member of the communication group receives/has access to all communication within the channel). According to one embodiment the method further comprises capturing a set of relevant systems associated with a worsening condition, based at least in part, on a patient assigned care plan, personal medical risks, and comorbidities. According to one embodiment, the method further comprises generating a symptom checker display configured to accept user reporting of any of the relevant symptoms. According to one embodiment, the method further comprises displaying the symptom check in conjunction with care plan tasks.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment,” or the like are not necessarily mutually exclusive and are intended to indicate that a particular feature, structure, or characteristic described in connection with the embodiment may be included in at least one embodiment. The appearances of such terms herein are not necessarily all referring to the same embodiment. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the invention. In the figures:

FIG. 1 is a block diagram of an example management, according to one embodiment;

FIG. 2 is a block diagram of an example system that can be augmented to perform the operations, processes, and/or functions disclosed;

FIG. 3 is a block diagram of an example system that can be augmented to perform the operations, processes, and/or functions disclosed;

FIG. 4 is a conceptual illustration of elements of a management system and the engagement that the system fosters between patients, educational and care content according to one embodiment;

FIG. 5 illustrates functions for building engagement, according to one embodiment;

FIG. 6 illustrates examples of interactions between the care team and patient user, according to one embodiment;

FIG. 7 illustrates an example display of readiness and recovery scores, according to one embodiment;

FIG. 8 illustrates a remote monitoring display, according to one embodiment;

FIG. 9 illustrates an example user interface that guides a user through a set of breathing exercises, according to one embodiment;

FIG. 10 shows example screen captures of a care plan display organized by activity, according to one embodiment;

FIG. 11 illustrates an example interactive engagement screen, according to one embodiment;

FIG. 12 illustrates an example screen capture of an interface showing daily progress rate for task completion, according to one embodiment;

FIG. 13 is a block diagram showing high level system component, supporting data, and user interface components, according to one embodiment;

FIG. 14 is a block diagram showing example data sets and models; according to one embodiment;

FIG. 15 is a block diagram showing example communication flows, according to one embodiment;

FIG. 16 is a block diagram showing high level components of a management system, according to one embodiment;

FIG. 17 is a block diagram of an example environment, according to one embodiment;

FIG. 18 is a block diagram of an example embodiment of a management system and execution flow, according to one embodiment;

FIG. 19 is a block diagram and example logic flow for matching between patients, ecosystem providers, support groups, and other resources, according to one embodiment;

FIG. 20 is a block diagram of example logic and descriptions of functional analysis employing the systems back and logic model and algorithms, according to one embodiment;

FIG. 21 is an example process for building an at a glance display, according to one embodiment;

FIGS. 22-29 show captures of example user interfaces, according to some embodiments;

FIGS. 30A-D are an example care plan data mapping, according to one embodiment;

FIGS. 31-38 show captures of example user interfaces, according to some embodiments;

FIGS. 39A-B show captures of an example user interface, according to some embodiments;

FIG. 40 illustrates example data transformations, according to one embodiment; and

FIG. 41 illustrates example processing of data transformations, according to one embodiment.

### DETAILED DESCRIPTION

According to some aspects, a management system is provided wherein the management system is configured to organize care execution and drive the best outcomes for all managed medical procedures by targeting and digesting educational material for patients in contemplating a procedure, preparing for a procedure and self after a procedure, as well as fostering engagement between the patient and their care team, so that questions and issues are resolved nearly immediately and any increased re-admittance risk can be flagged and resolved before such risks materialize into emergencies. In various embodiments, the system is configured to build and execute a care plan tailored to each respective patient. In some examples, the system provides customized user interfaces to respective patients via mobile applications or downloadable software. The customized user interfaces are tailored to limit the information a patient needs to review in order to understand and produce the best medical outcome for themselves. In further example, the user interface can be organized based on action items displayed to the user, and the mobile application can control presentation and execution of day-to-day action items to optimize compliance and deliver the best outcome. In one example, the system can organize the user interface according to compliance scores that are displayed in conjunction with elements of a care plan. The interface can be configured to dynamically emphasized and even re-organize displays based on compliance score. For example, trending scores of showing increasing non-compliance can trigger re-ordering of user interface displays, such that action items with compliance issues are displayed first and can also result in additional displays for accessing coaching or interacting with a patient's care team.

In further aspects, the system includes integrated dashboards for physician interaction and/or oversight by a care team. In some embodiments, a physician can log into the system and build a care plan for a respective patient based on filtered options that are identified, for example, using patient information, patient demographics, patient procedure, comorbidities, service provider, survey responses, etc. In various embodiments, the system is configured to generate and display a care team dashboard that captures and displays at a high resolution key clinical data, patient reported data and outcomes, and clinical notes. In some examples, the system can identify behavior patterns associated with patient activity, based for example, on inferring relationships between explicit data. In one example, the system can infer that larger supplies of patient medicine in a post-operative setting is required for a given patient responsive to feedback provided in the system. The inferred information can be developed automatically in response to identifying non-compliance of a patient in a region. The source of the non-compliance can be based on external factors including monsoon season, and the system can respond across patients and care plans to ensure a medicinal supply of sufficient volume across a broad range of matching patients. In various embodiments, the system can be configured to extrapolate a condition (e.g., monsoon season) to a match against broader groups of patients by extrapolating a geographic region impacted by such events and providing recommendations to change a care plan for approval and/or automatically adjusting a care plan to reflect an increased medicinal supply.

In still other embodiments, the system can be configured to analyze explicit care information (e.g. patient contacts, patient provider, patient procedure, procedure location, procedure provider, post care provider, etc.) and derive implicit relationships between elements of the explicit data. For example, the system can be configured to detail explicit care information in a graphical data structure. Various nodes of the data structure can be analyzed to generate inferred relationships and augment the graphical data structure. Such inferred relationships can then be used to augment care plans, individual action items, augment scoring of the action items, and the system can also use inferred relationships to improve patient compliance and/or outcomes. According to another aspect, the inventors have realized that conventional hospital based approaches cannot adequately engage with patients. The inventors have realized that the source of this problem stems from the fact that the majority of patient care happens outside the hospital walls. Thus, there is a need for a system that integrates pre-procedure preparation and compliance with the same, optimizes treatment compliance, and further provides for post procedure treatment and regimens that can be easily followed with feedback that can be easily understood by any patient. The inventors have also realized that there is a need for a system that can better inform treating physicians and provide insight into post procedure regimen and compliance. Various embodiments of the management system address at least some of these outstanding needs and issues.

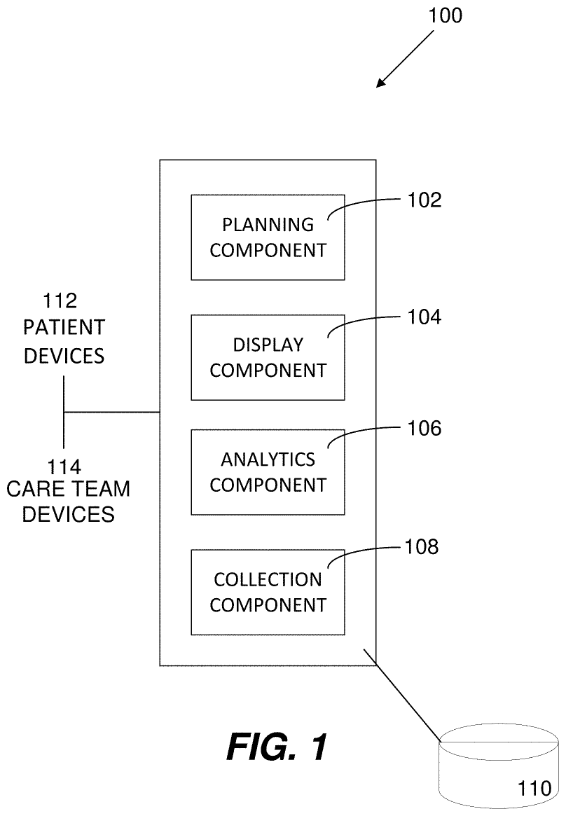

Shown in FIG. 1 is a block diagram of an example management system 100. The management system 100 can include respective components that are executed by the system to provide specific functionality, and can also execute any of the discussed functionality without instantiating the respective components. According to one embodiment, the management system 100 operates a central service and/or system. The management system 100 can be a standard server or can be virtualized, and can also be executed on cloud architecture.

According to one embodiment, the system includes a planning component 102. The planning component can be configured with any number of treatment plans for execution. The planning component is configured to establish each treatment plan or care journey as a series of individual steps that are and/or can be tailored to a specific patient that brings the patient from pre-treatment processes and/or preparation to execution of the actual treatment, as well as the post-treatment processes and/or follow up. In various embodiments, the planning component can be configured to schedule the timing for each step to be performed in a given day to optimize patient compliance. For example, the system can monitor activity by the patient and determine when specific events occurred based on activity information collected by a user device and/or remote monitoring devices connected to the user device. Scheduling medication to occur in compliance with any dietary requirements (e.g., take on empty stomach, take with food, drink with water, etc.) simplifies the process for patients and eliminates execution error during pre-treatment, treatment, and post-treatment phases of a patient care journey.

In further embodiments, the planning component can interact with a display component 104 configured to generate displays on an end user device (e.g., 112 or 114) associated with each step of a care journey. The planning component can be configured to break a care journey down into discreet steps, and the display component 104 configured to tailor displays (e.g., including integrating instructions videos on how to accomplish the discreet step) to the associated step and/or the user.

In further embodiments, the system 100 can include an analytic component configured to analyze incoming treatment data from end users. In one example, patients can be paired with remote health monitoring devices and data collected during pre, post or treatment phases. In some examples, the system and/or analysis component 106 is configured to score the user's execution of the steps in a care journey. The scores can be integrated into the user customized display, and recommendations and/or suggestions can be presented to the user on how to improve scoring. In some examples, low scoring can trigger further customization of a care plan to a user (e.g., so activities occur in periods of time where the user is more likely to complete them). In other embodiments, additional instructional displays can be introduced into the user interface displays responsive to low scoring. And in yet other embodiments, remote coaching interfaces can be displayed more prominently in the user interface displays to further encourage better compliance. In one embodiment, remote coaching sessions can be automatically executed (e.g., responsive to low scoring) at the time the user is scheduled to perform a step in their care journey/treatment plan.

The automatic triggering by the system eliminates any technical barrier (e.g., of the user) to engaging the system and/or the best advice on obtaining health goals.

Various embodiments of the system can also include dedicated collection component(s) 108 for collection of remotely monitored health information. The collection component 108 can be configured with additional security to ensure safe and private collection of health data. The health data can be used by the analysis component to create treatment scores, identify potential issues, adjust treatment plans and/or execution specifics, and further can be used to re-organize day to day steps managed as part of the treatment plan.

In some embodiments, as data is collection and/or stored in database 110, the system can learn from collected execution information to adjust step by step organization, particulars of execution (e.g., time of day, etc.), and/or customize the presentation of the steps to the end user, among other options. Collected data can also be analyzed (e.g., by component 106) by surface indicators and sent to a care team. For example, indicators associated with complications can be immediately recognized by the system and updated care plans executed to manage the potential complication. In other embodiments, a care team can be made aware of potential issues long before they manifest, permitting the care team to adjust treatment or management accordingly.

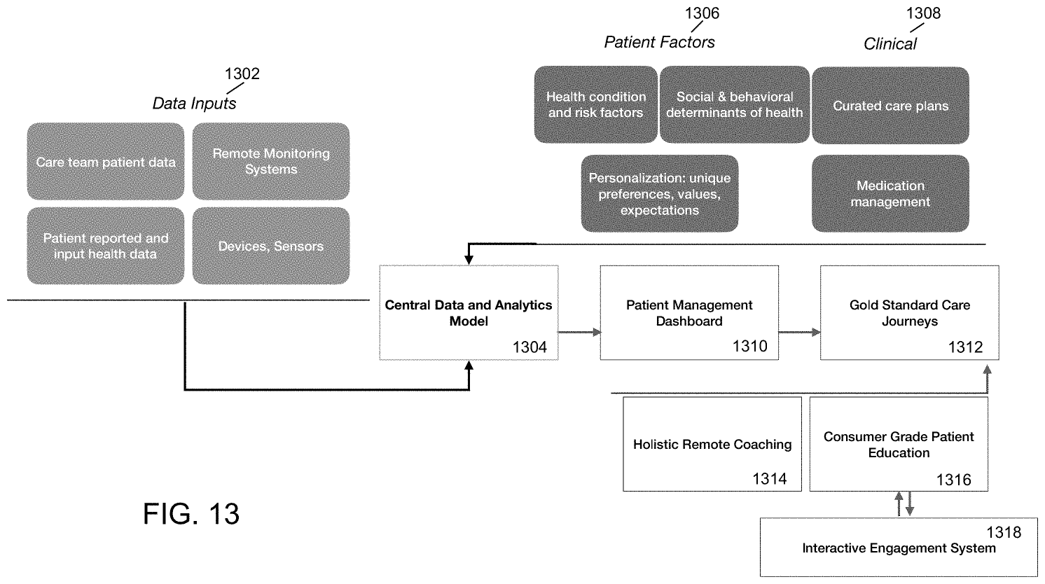

FIG. 13 is an example block diagram 1300 of high-level system components, data, and interface elements according to one embodiment of a management system. According to various embodiments, management system can expect to receive a variety of data inputs 1302. For example, the data inputs 1302 can include care team patient data, remote monitoring data, patient reported information, data captured from monitoring devices and/or sensors. According to some embodiments, the various data inputs (e.g., 1302) can be used by central data and analytics module 1304 to develop care plans for respective patients (e.g. 1312). In some examples, input data can include information on patient factors 1306. For example, the patient factors can include health condition and risk factors, social and behavioral determinants of health, unique preferences, values, and expectations of respective patients (e.g. reflected in user profiles and/or preference settings, among other options.). In further example, input data can also include clinical information (e.g., 1308) associated with, for example, curated care plans, medication management information, among other options.

According to one embodiment, the analytics module 1304 is configured to process the various data inputs and provide visualization of relevant metrics in a patient management dashboard (e.g. 1310). According to one example, the system is configured to aggregate and filter information to facilitate development of care plans for respective patients.

According to one embodiment, a care team member can review a patient management dashboard (e.g. 1310) and use the information provided to develop a care plan for respective patient at 1312. In various embodiments, the care plan can include a variety of information sources. For example, the care plan can include options for remote coaching (e.g. 1314), patient education material (e.g., 1316), and functionality for interactive engagement (e.g., via interactive engagement subsystem 1318).

In further embodiments, the central data and analytics module (e.g. 1304) is configured to collect data during execution of the care plan and update action items, coaching options, education information, interactive functions, among other options as additional information is collected on a patient care journey. In some instances, the system can alter a care plan (e.g. reorder action items, reschedule action items, trigger automatic coaching sessions, among other options) in response to determining noncompliance or issues with execution of the patient care plan. In some examples, alteration to a care plan can include changes in nutrition education and/or nutrition recommendations provided to a patient and may also include changes in educational material provided to a patient to facilitate their compliance with the care plan. In further embodiments, the management system can be configured to identify issues with care plan compliance and/or action item compliance to identify in real time risk associated with the re-admittance or further medical issue and trigger intervention by a care team responsive to a risk threshold.

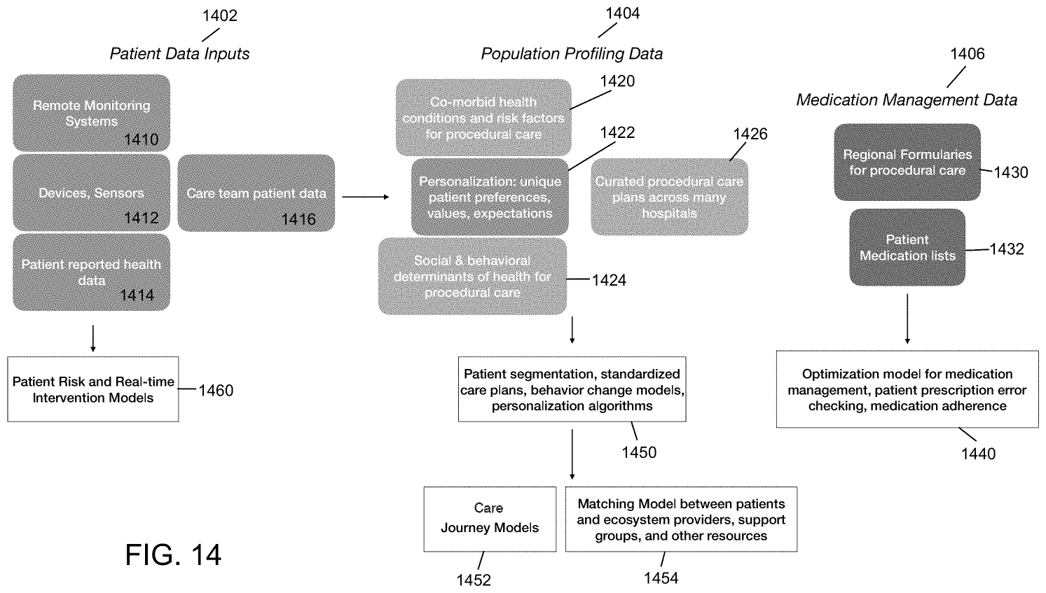

FIG. 14 illustrates example data sets and models that can be used by management system to evaluate patient compliance and/or risk and/or real-time intervention modeling. According to some embodiments, the data sets can include patient data inputs 1402 received from or about a patient executing a care plan. For example, a patient can be issued remote monitoring systems (e.g., 1410), devices and/or sensors (e.g. 1412), or the system integrate with devices and/or sensors available to a patient to provide at least some of the patient data input at 1402. In one example, the system is configured to collect health data from a patient as part of execution of an action plan. For example, an action item in a care plan can include a question and in response information is presented to the user in the user interface. User responses can be collected by the system and form part of patient data inputs 1402. In addition, the patient data inputs can include care team patient data. In various embodiments, the patient data inputs can be used to evaluate patient risk (e.g., for re-admittance and/or health complications - which can be reflected in patient risk in real time intervention models 1460). In some embodiments, the patient data inputs can be used to train machine learning models to recognize patterns that are associated with re-admittance risk and/or additional health issues.

According to some embodiments, the system can also analyze population profiling data to tailor care plans and/or optimized risk evaluation models. For example, co-morbid health conditions and risk conditions for procedural care can be evaluated as part of population profiling data (e.g. 1420). In further example, personalization information can be captured and/or entered by users. For example, a user can specify unique patient preferences, values, and/or expectations that can be evaluated as a factor in developing a care plan and/or identifying risk factors that affect intervention models. In addition, social and behavioral determinants of health (e.g. 1424) can be incorporated into patient and/or population profiling data in further use for identifying risk factors and/or adjusting machine learning models. In another example, curated procedural care plans (e.g. 1426) can also be used as part of developing profiling data for user population.

According to further embodiments, another data set and model for analysis can be derived from medication management data (e.g. 1406). For example, regional formulary for procedural care can be used to optimize and or select action items for care plan (e.g. 1430). In another example, patient medication lists (e.g. 1432) can be accessed to determine daily action items that should be included in a care plan.

In various implementation, the system can be configured to resolve issues associated with medication administration and, for example, generate an optimization model for medication management, patient prescription error checking, and/or medication adherence (e.g. 1440). Any one or more of the foregoing factors can be used in developing action items for a care plan (e.g. daily action items or required action list elements, etc.).

According to various embodiments, any of the data sets can be used to optimize selection of patient care journeys and/or to augment action items within a care plan. In further example, the data sets can be used to facilitate patient segmentation, filter selection of care plan elements, to develop a standard set of care plan options to present any user interface responsive to matched profile information, establish baseline behavior change models (and patterns for matching behavior change indicative of increased risk), and develop personalization algorithms for respective patient care plans (e.g., 1450). According to some embodiments, the results are optimized care journey models (e.g. 1452), and/or options for matching models between patients and providers, support groups and other resources (e.g. 1454).

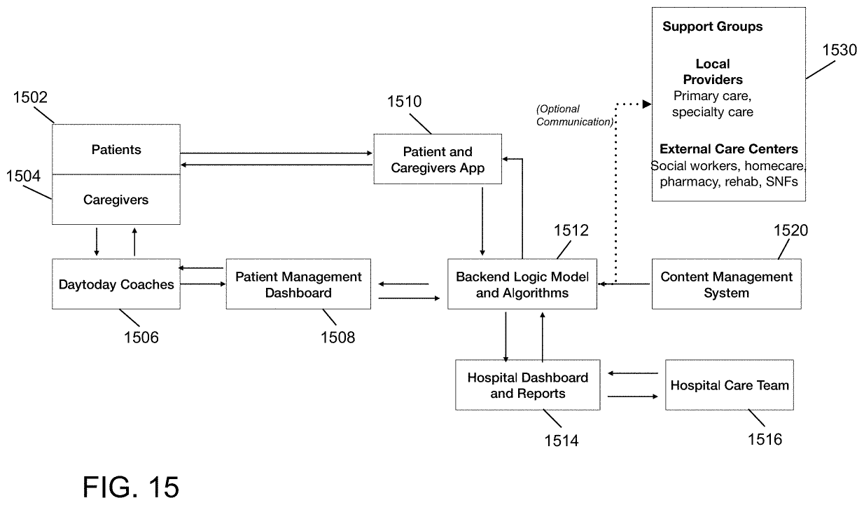

Shown in FIG. 15 are example communication flows between system components, users, etc. according to one embodiment patients 1502 and caregivers 1504 can interact with management system, and further interact with system-based coaching functions at 1506. Patient input data and/or data monitored on given patients can be communicated via a patient and/or caregiver's application 1510 to the system. According to some embodiments, the system can process the received data based on logic models and rule-based algorithms (e.g., 1512). Information may also be received from coaching interactions (e.g. 1506) which can be displayed in a management dashboard 1508. Further embodiments provide options for care team members and/or coaches to input additional information into the management dashboard which can also be communicated to various analytic components (e.g. 1512).

The analytic components are configured to also receive data from additional systems and/or sources. For example the analysis components are configured to receive information from various hospitals and/or operatives locations that provide procedures to subscribed patients. In one example, the hospital care team (e.g. 1516) can access their own system and/or dashboards and reports (e.g. 1514) and provide that information to the system for analysis. In further example, a hospital care team can provide their notes, suggestions, analysis, diagnoses, and other relevant information that can be incorporated and/or analyzed by analytic components of the system.

Other system tie-ins can include links to content management systems (e.g. 1520). According to one embodiment, the content management system 1520 is configured to provide educational information associated with care plans and/or action items within a care plan. For example, the content management system can be the source of your breathing exercise display presented in the user interface to a patient during a care plan execution. In other embodiments, educational information can include content from 1520 that is tailored to a specific task a patient needs to execute.

In some embodiments the system and/or analytic components can include optional communication pathways to facilitate patient support and enable better integration between the patient, their care journey, and long-term care goals. For example, the system can include optional communication pathways to external care centers. The external care centers (e.g. 1530) can include social workers, home care, pharmacy, rehab centers, etc. In further embodiments, support groups can include local providers. The local providers may be primary care physicians, specialty care physicians, and/or other treatment facilities.

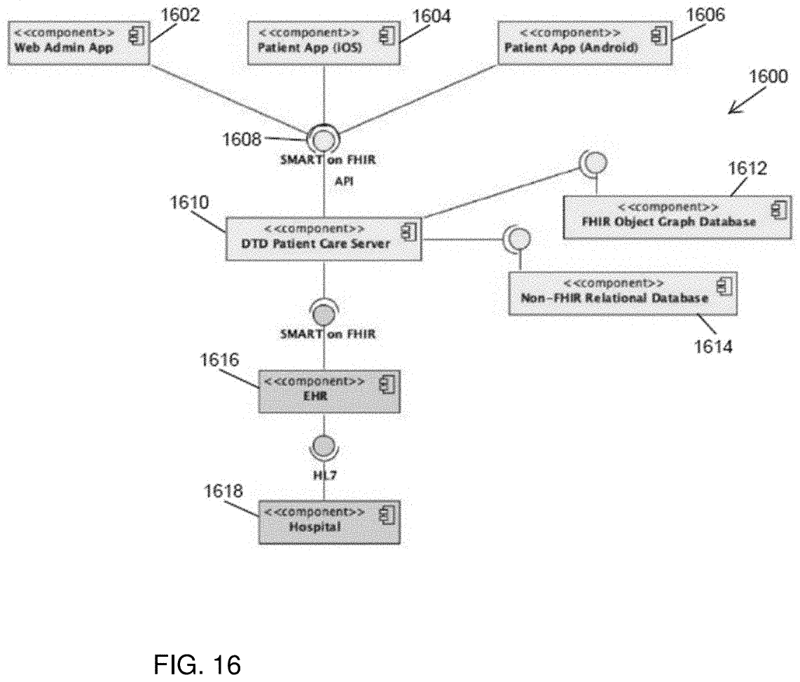

FIG. 16 is a block diagram showing high level components of a management system 1600. According to one embodiment, downloadable software (e.g., mobile application) can be distributed to end user populations. For example, a web admin application 1602 can be used by care plan administrators/care team members to access the system, develop tailored care plans, mange execution, and/or trigger intervention among other options. In another example, a patient application 1604 is configured to provide patient access to respective care plans, day to day activity displays, track user compliance with activity items, prompt users to meet activity goals, remind users of activities, schedules, itineraries, etc., to support care plan execution and/or to trigger intervention and/or coaching functions. In further examples, the applications can be made available in a variety of formation (e.g., iOS at 1604, ANDROID at 1606, among other options, including desktop applications).

The various applications can connect to a patient care server (e.g., 1610) via an application programming interface (API). In some examples, the system includes a SMART on FHIR compliant API that manages data interchange between components. According to one embodiment, patient care server (e.g., 1610) operates as the backend server for various applications that manage care plans, patient engagement, and care team interactions. The server can include an API conformant with the SMART On FHIR standards. The various specification, data models, authentication, etc. are incorporated here by reference. According to various embodiments, the web admin app 1602 can be a react application, where web pages need not be rendered by server.

In some setting the server 1610 interoperates with external electronic health record (EHR) systems (e.g., 1616). For example, these interactions are managed under the SMART On FHIR standard. In one example, the web admin app 106 can be launched from within the EHR system 1616, and patient registration for the management system can include selecting EHR patients that are to be enrolled as system registered patients. EHR integration can include a connection to hospital systems (including for example, hospitals at which patients are scheduled for procedures. Communication can be managed between EHR component 1616 and hospital systems 1618 under the HL7 standard, propagated by HL7 organization, which is incorporated by reference herein.

In further embodiments, patient data can be read from the HER systems but not copied to preserve confidentiality in protected health information (“PHI”). In various embodiments, the system is configured to create new patient resource records linked to the patient resource provided by the EHR. In further embodiments, data persistence is implemented with an object graph database (e.g., 1612) or other non-relational format database (e.g., document based model, etc.). For example, the system can employ a Neo4J object graph to capture and store patient information. In additional to FHIR based data, the system can include a relational based database that specifies user passwords, uploads documents, etc., which may be stored in a Postgres relational database.

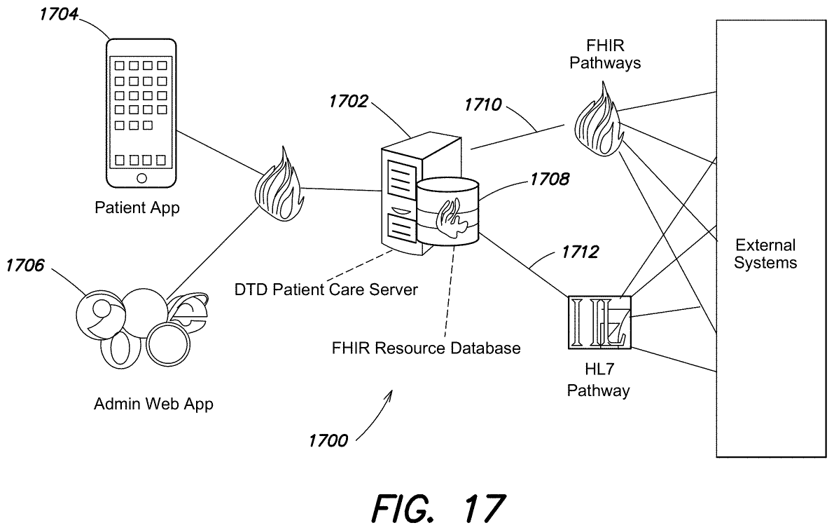

FIG. 17 is a block diagram of an example environment 1700. According to various embodiments, patients can be registered to access a central server 1702 via their own mobile devices 1704. The registration functions can be executed using a web based administration application (e.g., 1706). The central server can be configured to build a database (e.g., 1708) of resources defined according to the FHIR architecture. In further embodiments, the server is configured to connect to various external systems via communication pathways (e.g., 1710 and/or 1712). The server may do so to capture information and translate the information into the server database architecture (e.g., a FHIR compliant graphical object database). Additionally, the various communication pathways can be used to provide information on procedures being performed for registered patient and/or to receive information on such procedures. In further example, the pathways can be used to connect to prescription servicers, counseling services, and other external systems (e.g., CARECLOUD, ALLSCRIPTS, EPIC, CERNER, etc.).

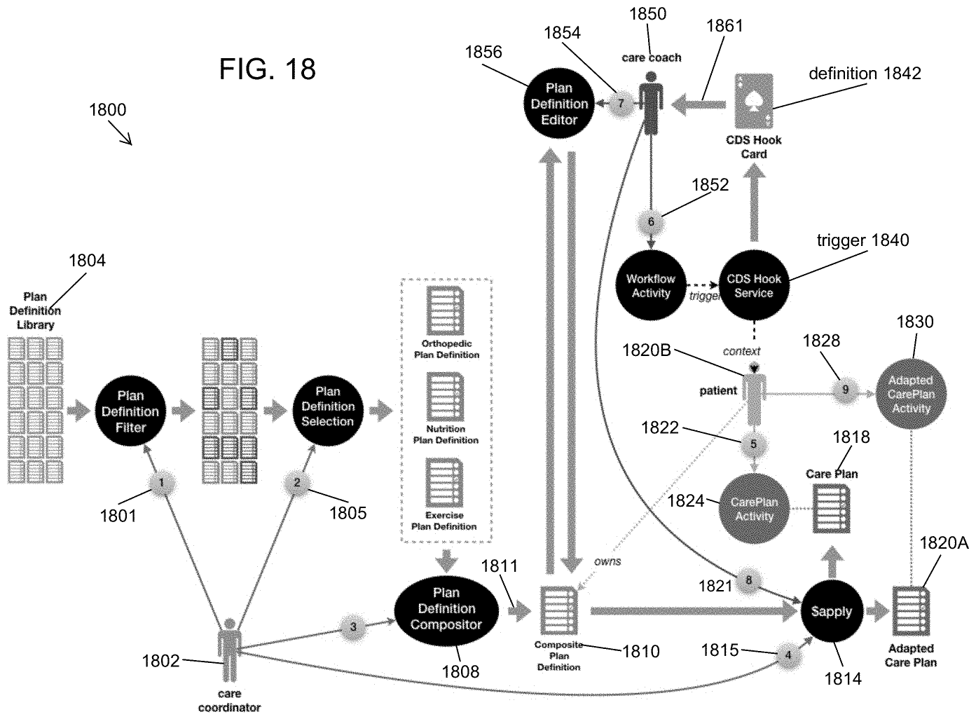

FIG. 18 is a block diagram of an example embodiment of a management system and execution flow 1800. At 1801 a care coordinator 1802 (e.g., member of a care team and/or care administration) can access a web admin tool (“WAT”) to register a patient on the management system. As part of registration, the system enables entry of patient context information and creation of a patient record information. For example, the coordinator 1802 can establish the care context that includes the patient's provider, procedure, physician and comorbidities, among other options. According to one embodiment, the context information is used to access a plan definition library 1804 including plan definitions (e.g., records of procedures, treatment, interactions, steps needed to complete recovery actions, exercised, dietary procedure, etc.) and filter the library to those plan definitions that match the patient context. According to one embodiment, a coordinator can then select from a filtered set of plan definitions at 1805. In other embodiments, the system can be configured to filter and select plan definitions automatically based on the filter set of plan definitions. The plan definitions can include exercises for a patient to perform, nutrition actions for the patient, preparation actions for the patient, among other options (e.g., shown at 1806). In various embodiment, plan definition can incorporate patient profile information to establish tasks and/or defined specific actions. In one example, nutrition planning can incorporate a cultural component and adjust nutrition selections accordingly. Once the plan definitions are selected, a plan creation component 1808 (e.g., plan definition compositor) can be configured to execute. Once executed, the component 1808 is configured to merge any selected plan definitions. As part of merging, the component can identify any inconsistencies, conflicts, and/or incompatibilities. The system can be configured to resolve these issues or present the conflicts to the coordinator 1802 for resolution. Once the plan is established and/conflicts resolved, a composite plan definition 1810 is established at 1811, and can then be assigned to a patient. Additional operations can translate the established plans into specific actions and/or schedules tailored to a given patient. For example, the system and flow can include and apply operation 1814 at 1815 that results in a care plan 1818 and/or an adjusted care plan 1820A. In various embodiment, system feedback, recommendations and/or analysis can be used to adjust a care plan, steps, timing, exercised, nutrition etc., and feedback during execution can be used to create an adjusted care plan 1820A.

For example, as the patient complies (or not) with their care plan, two separate mechanisms come into play that can influence modifications to the care plan. Such modifications are intended to improve the probability of more favorable patient outcomes than the currently active care plan. The first mechanism is a regularly scheduled assessment process undertaken by the patient's care coach. The purpose of the assessment is to determine the patient's level of compliance and progress. Based on the results, the care coach may choose to create a modified plan definition specific to the patient and generate a new (adapted) care plan from it. The second mechanism uses CDS Hooks, which inform the care coach about changes to a patient's condition that would warrant revisiting their care plan with the aim of possibly adapting the plan definition and generating a new (adapted) care plan from it. CDS hook can include a variety of automated monitors that, for example, capture and analyze health information for indications of improving, worsening, conditions, stable conditions, etc. In additional the CDS hooks can be configured to monitor patient reported information to determine if intervention or changes are warranted. The CDS can generate recommendations for needed changes and/or automatically adjust elements of a care plan.

According to one embodiment, a care coach can use the Plan Definition Editor to revise the Care Plan Definition (CPD), then approve the revised CPD and instructs the WAT to create a new, adapted, care plan for the patient by performing an $apply operation on the revised CPD. Once approved, the patient then engages with activities prescribed by the adapted care plan.”

According to some embodiments, the patient can interact with and perform the activities defined in the plan 1818, for example, at 1822 the patient can execute any activities 1824 specified in the plan 1818. Additionally, the patient may perform any advanced care plan activities (e.g., 1830) at 1828. In some embodiments, care plans can be changed during the course of execution. In one example, a patient's care coach 1850 performs some work activity on their admin tool which can execute a trigger 1840 (e.g., an executable associated with an action/activity on the system). The service is configured to use the patient as a context and applies a set of predefined rules against this context to deliver the information gathered by these rules and send an information object (e.g., a “card”) to the care coach 1850 at 1861. According to some examples, the rules can be configured to evaluate the patient's compliance with their care plan as well as their current situation. If the card indicates that the patient's situation has changed such that if the care plan were generated now it would result in different or modified activities, then the care coach uses and editor 1856 at 1854 (e.g., a plan definition editor (PDE)) to edit the patient's CPD. In other embodiments, the system can identify the difference between the original plan and any modifications and merge the changes into the patient plan.

According to some embodiments, the care coach 1850 approves the revised plan (e.g., 1821) and triggers creation of a new care plan for the patient (which can include an apply operations 1814 to output an updated plan 1820A).

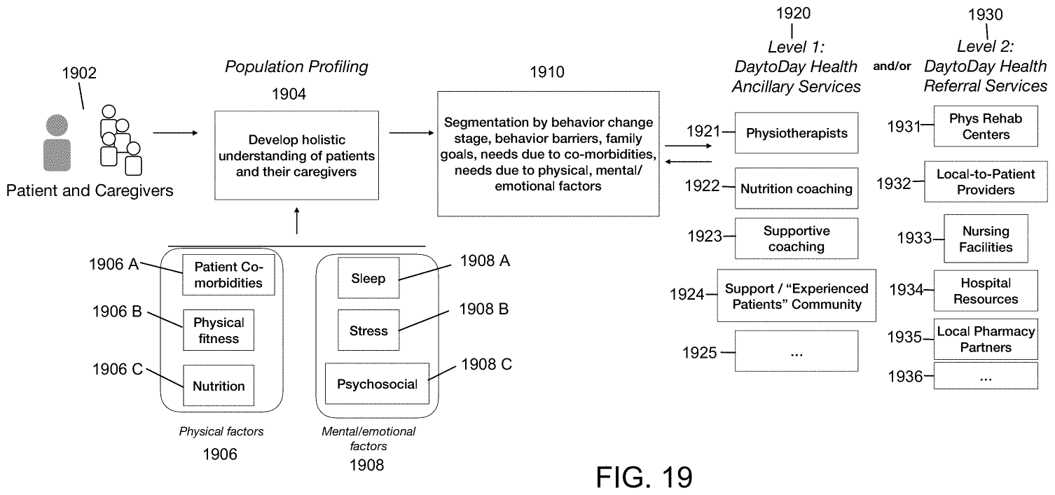

Shown in FIG. 19, is a block diagram and example logic flow for matching between patients, ecosystem providers, support groups, and other resources. For example, patients and caregivers (e.g., 1902) can access the system and utilize the day-to-day planning and execution services. According to some embodiments, the management system and/or analytic components can develop patient profiles (e.g., 1904) to support tailored care plans. The patient profiles can enable development of holistic models and facilitate the understanding of patients and their caregivers. In some examples, the system and/or analytic components can be configured to analyze a number of physical factors, mental/emotional factors, among other options to develop holistic patient profiles. For example, physical factors that are integrated into the patient profile can include patient co-morbidities (e.g., 1906A), physical fitness characteristics (e.g., 1906B), nutrition information (e.g., 1908C), among other options. In another example, mental/emotional factors can include sleep factors (e.g., 1908A), stress factors (e.g., 1908B), psychosocial factors (e.g., 1908C), among other options. In some embodiments, machine learning models can be trained on any combination of the above factors to predict potential treatment issues and/or to identify indicators of potential issues in treatment and/or plan compliance.

According to various embodiments, developing of patient models and/or patient profiling can include analysis to develop segmentation by behavior change stage, family goals, needs due to co-morbidities, needs due to any physical, mental/emotional factors, among other options.

According to various embodiments, behavior change stages can be defined by a predetermined model combining various evidence-based behavior change theories, for example the Trans-theoretical Model of Change which includes stages of pre-contemplation, contemplation, preparation, action, and maintenance, and Self Determination Theory where individual needs of competence, relatedness, and autonomy must be fulfilled to motivate an individual to change. This model is created by having patients complete validated, standardized assessments and then combining the results into a composite to be part of their personal profile. Patients can then be segmented by the resulting composite, multi-faceted stages of change to determine the combination of care management services they need, as well as core coaching and educational content.

In some examples, patterns within behavior change stage, behavior barriers, needs due to co-morbidities & physical mental/emotional factors can be developed within graphical representations and inferred relationships can be reflected in associations between nodes in the graph. In other examples, machine learning models can be used to analyze behavior and outcomes associated with patient populations, and then applied to predict likely results and/or issues associated with similar patient populations.

According to some embodiments, the patient profiling can be used by the system to develop referrals and/or requests to additional services. According to one embodiment, the management system can provide access to ancillary services (e.g., 1920). For example, various execution plans may include services provided by physiotherapists (e.g., 19 21), nutritionists (e.g., 1922), supportive coaching (e.g., 1923), support groups and/or experienced patient community members (e.g., 1924), among other options, shown for example at 1925. Various services can be segmented into a degree or level of an intervention required. For example, services provided by physiotherapists, nutritionists, support groups, etc., can be organized into a level I ancillary services group. A level II services group may require referrals in order to engage in the activity. In some embodiments, the management system is configured to enable referral to additional services as needed (e.g., 1930). For example, the management system can integrate and/or request services from a physical rehabilitation center (e.g., 1931), providers proximate and/or local to a patient (e.g., 1932), nursing facilities (e.g., 1933), hospital and/or hospital resources (e.g., 1934), local pharmacy partners (e.g., 1935), among other options shown at 1936.

According to some embodiments, patients can be matched to level I and/or level II services depending on the severity of their needs and whether the physical presence of the patient is necessary for a desired outcome. In various embodiments, the management system is configured to model a patient's current condition and contacts and confirm whether additional services may be required in order to improve compliance, outcome, etc.

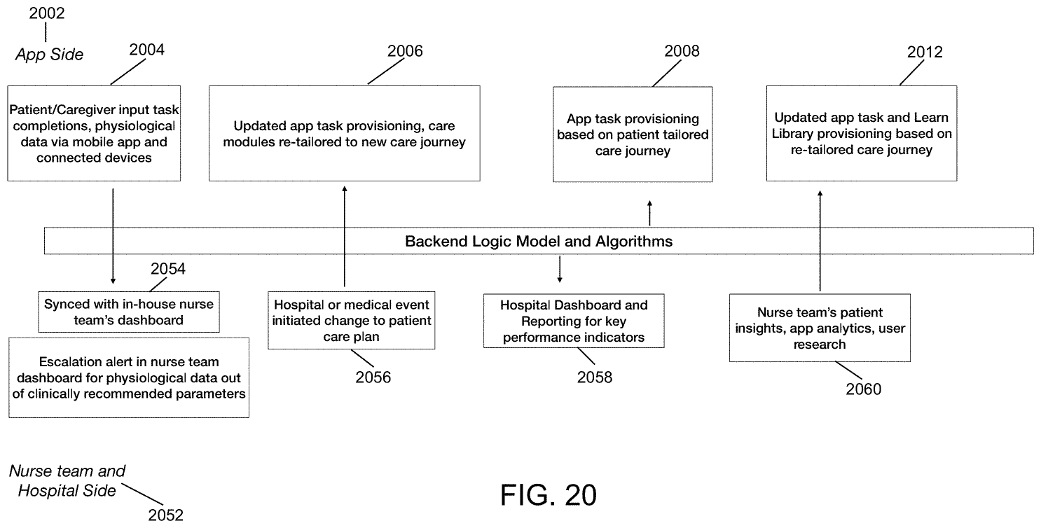

Shown in FIG. 20 is a block diagram of example logic and descriptions of functional analysis employing the systems back and logic model and algorithms. According to one embodiment, users on the application side 2002 include patients and caregivers who execute various tasks specified in a care plan to provide physiological data and other inputs (e.g., via a mobile app and/or any connected devices). As the users on the application side execute their tasks, the system is configured to provide access to the compiled information, for example, the data can be synchronized with a care team dashboard (e.g., an in-house nurse or care team member's dashboard at 2054) available at a care team or hospital accessible side of the system (e.g., 2052).

According to some embodiments, patients and/or caregivers can receive updated application information including task provisioning, care modules, dynamically adjusted tasks, and/or updated care modules that are tailored to a new care journey, among other options. For example, the management system can be configured to adjust care plan execution (e.g., timing, scheduling, task, task parameters, etc.) responsive to detecting issues associated with care plan execution (for example, machine learning prediction of poor outcome or prediction of decrease in compliance with task execution, among other options). In further embodiments, the care team facing functions provided by the management system can provide an indication of a hospital or medical event initiated change to a patient care plan. Various embodiments provide the ability to immediately change an already defined care plan responsive to new hospital and/or medical events enabling the transition to a new approach in a manner not available on conventional approaches.

In further embodiments, the management system itself can identify issues and/or predictions of issues for a patient or group and develop actions that automatically resolve the potential issue. In still other examples, the management system can identify potential or likely issues and surface those to the care team with recommendations on resolutions that can be adopted by the care team members. For example, the management system can update a hospital dashboard and reporting interfaces for key indicators and highlight potential or predicted issues within the dashboards or reporting interfaces. In one example, the management system provides visualization of key performance indicators and delivers predictions on patient outcome, compliance, and other factors in the dashboard displays. The system may also define change thresholds that are used to generate alerts and the system can be configured to change the interface to highlight potential issues and potential resolutions for review by a care team (e.g., the hospital dashboard and reporting interfaces at 2058). In one example, the system can reorder the dashboard display shown to care team members, or only display a key performance indicator that is associated with an issue, until the information is acknowledged, among other options.

According to some embodiments, patients and/or caregivers are able to view care plan tasks in a mobile application and provisioning of tasks can occur based on a care plan tailored to a respective patient. For example, tailoring can include medication distribution cognizant of the patient's ability in proclivity to obtain and follow a medication regimen. In one example, the system can identify significant weather conditions as a potential issue and cause for noncompliance. In further example, a monsoon season may be a significant indicator that a larger than normal medicinal supply will be required in order to ensure compliance with the dosing regimen.

In further embodiments, not only can the tasks and care plan be tailored to a respective patient but also a learning library and instructional information provided to the patient through the application can be tailored specifically to the respective patient. In addition, any changes in the care plan or indications of issues that may be likely or predicted can also be used to update the application display and/or to tailor a patient learning library to resolve the specific issues (e.g., 2012). According to some embodiments, the management system can be configured to analyze a patient care journey, compliance indicators, potential issues, and/or predict likely problems or outcomes based on care journey execution. In addition, a care team and/or care team member can review the data captured on an ongoing basis to provide insights, application analytics, user research, among other options all of which can be employed to update application tasks, patient learning library, and/or to retailer a care plan for respective patient (e.g., at 2012).

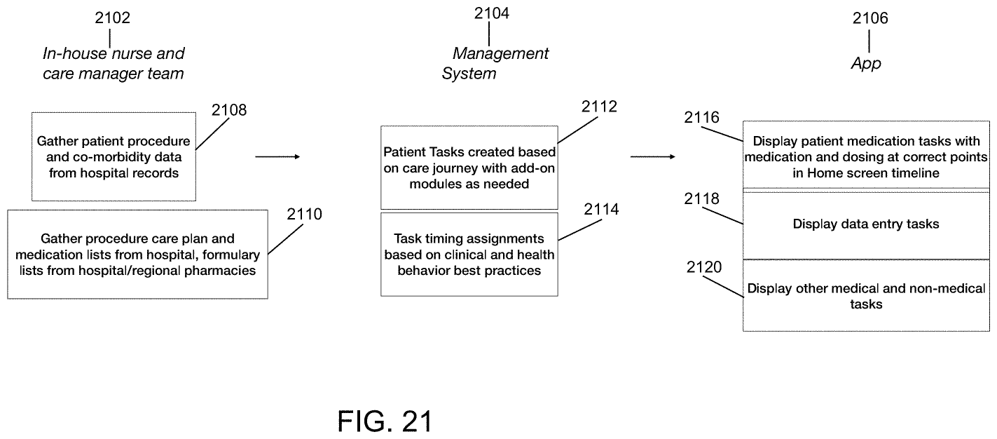

FIG. 21 shows an example process for building an at a glance display shown to an end user. For example, a care team member (e.g., in-house nurse and/or care manager) can gather patient procedure and co-morbidity data from hospital records through the management system (e.g., 2104) and/or gather procedure care plan and medication lists from hospital, formulary lists and/or from hospital/regional pharmacies, among other options (e.g., 2108-2110).

As discussed above, once patients are registered and/or patient contact information is available on the system, patient tasks can be created based on the care plan defined for the respective patient. In some examples, a tailored care plan may require additional services and/or add-on modules that can be provided to a specific patient and/or application (e.g., 2112). In further embodiments, task and timing assignments can be based on clinical and health behavior best practices to establish an initial execution and timing (e.g., 2114). In further embodiments, patient profile information can be used to adjust task execution, task ordering, task timing, among other options.

According to various embodiments, once a care plan has been defined the patient and/or caregiver can access their day to day execution plan which will display patient medication tasks, the medication to take, the dosing for the medication, and establish the appropriate timing and any requirements taking the medication (e.g., fast for two hours, take with food, among other options) at 2116. Patients and or caregivers may also access any data entry tasks (e.g., 2118) via their mobile application. Each care plan may be displayed with other medical and nonmedical tasks that will help the patient on their care journey (e.g., 2120).

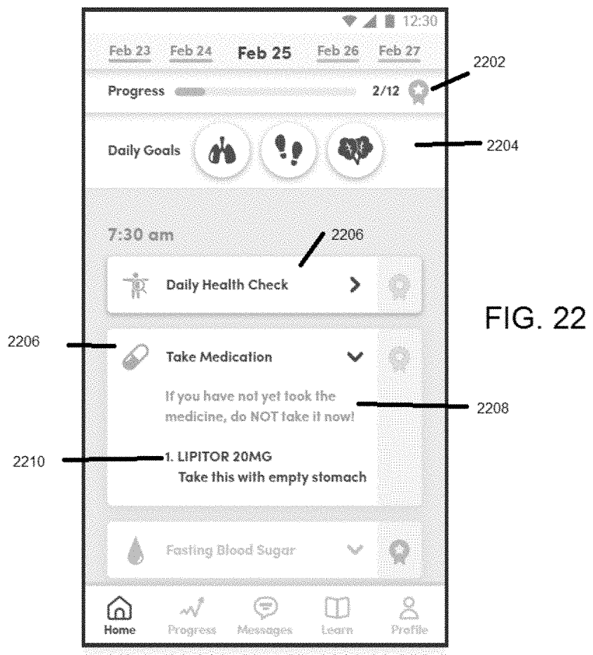

Shown in FIG. 22 is an example user interface according to one embodiment. The user interface organizes a patient's activities into daily tasks and scheduled times. At 2201 a patient can select other days, however, the default is a current date and a current set of tasks that are shown organized based on time of day. In some embodiments, the user interface includes a progress indicator (e.g., 2202) to provide an easy visualization for completion tracking. Based on completed tasks, users may be provided visual indicators and/or badges showing completion of daily goals. For example, at 2204 the user interface can include displays for accomplish goals (e.g., completed breathing exercises, achieved step count, among other options).

Shown in the UI, is a task at 7:30 AM for completing a daily health check at 2206. Selection in the UI of the daily health check may trigger display of a survey requesting information on symptoms relevant to the patient's procedure and/or condition. For example, the patient may be asked to report on chest pain, heart rate, racing heart rate, difficulty breathing, fever or chills, any lower extremity swelling, among other options. In another example, additional survey requests may be made for patients who underwent a surgical procedure. According to one embodiment, the patient may be asked if they are experiencing any pain at their incision site, if they observe any increased redness at the incision site, and/or if there is any swelling or drainage at the incision site. In further embodiments, the survey questions are tailored to the specific procedure and/or operation relevant to the given patient. Positive responses to the questions in the survey can trigger intervention functionality, which can include, for example, communicating alerts to a care team or care team member.

Shown in 2206 is another task displayed to the patient. In this example, the task includes taking medication. In some embodiments, tasks may update themselves based on whether or not completion has occurred according to the scheduled timing. For example at 2208, a warning message may be displayed to avoid complications. In this example, the care plan instructions provide information to a user that if they have not taken their medication by the allotted time they will obtain a better medical outcome by not taking the medicine at a late time. Conventional systems simply fail to provide tailored information and fail to advise a patient on day-to-day tasks in an integrated and optimized for best outcome manner.

In addition to the task indicator, the user interface can include specification of the medicine to be taken (e.g., 2210). In further example, the user interface may be configured to take the user to an image of the drug they are supposed to be taking. For example, the user may click on the indication “Lipitor 20 MG” to be shown an image of the pill or medication that should be taken.

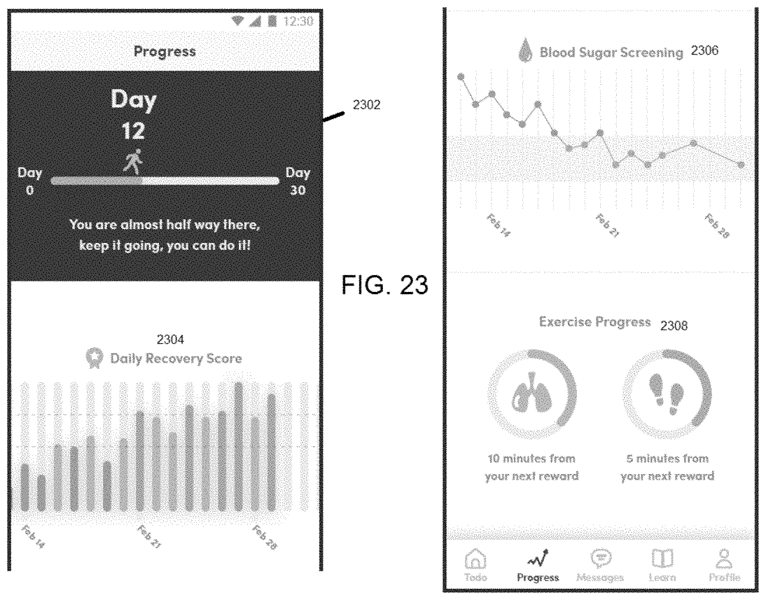

Shown in FIG. 23 is an example progress display for an overall patient care plan. At an upper portion of the user interface, shown is a progress indicator reflecting the number of days in care plan (e.g., 2302). At 2304 on a graph of daily recovery scores can also be shown. The visualization of daily recovery scores summarizes information on daily progress and provides direction to patients on potential needs to improve compliance and ultimately outcome for their rehabilitation. In further embodiments, the user interface can include information on respective activities and/or tests. For example, at 2306 the UI can display information on the patient's blood sugar screening. As shown in 2306, initial levels for the patient's blood sugar were highlighted but decreasing until they fell within a desired range shown by the highlighted portion 2306.

According to further embodiments, summary information for the tasks and care plan can also be provided in the user interface. For example, exercise progress can be shown at 2308. Any activity associated with specific exercise progress or tasks can also be linked to rewards and/or badges for completing various thresholds. For example, shown at 2308 is a progress indicator and the textual display to indicate how much more time and/or effort would be required to meet the next goal. By summarizing the information into an easily understood user interface element, the system limits the amount of information the patient must consume and/or understand in order to improve their own outcome and/or improve compliance with their care plan.

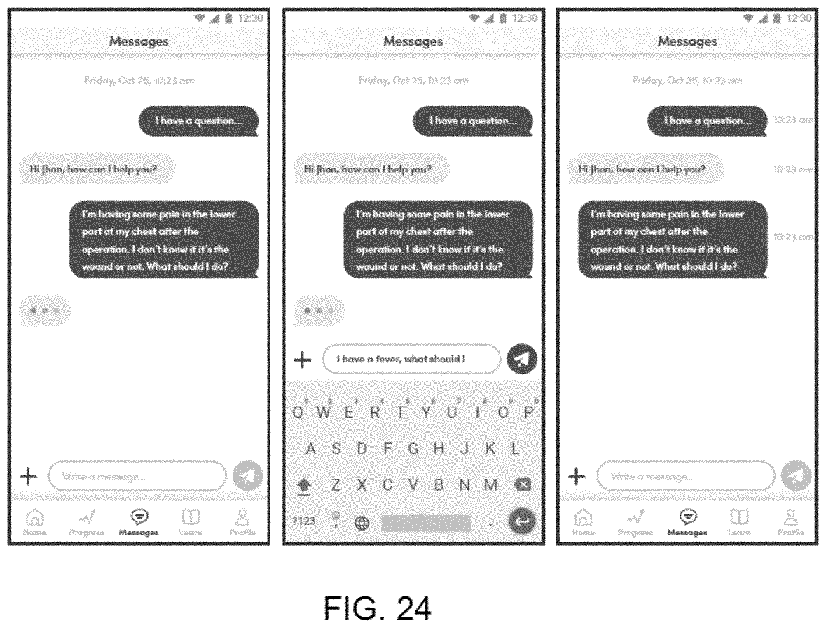

FIG. 24 shows a messaging interface made available through patient or user application. According to some embodiments, the messaging interface can be triggered automatically responsive to information provided in a care survey (e.g., having chest pains). In addition and/or in the alternative a patient can access the messaging interface simply by selecting “messages” at the bottom of the user interface. Once activated the system is configured to connect the patient to a member of their care team and/or a qualified practitioner who can handle the medical issue being requested. For example, a patient can ask questions about their procedure, their recovery, their current symptoms, among other options, and receive immediate feedback as to steps to take to secure a best outcome. By simplifying the technical requirements for patient access to their provider and/or relevant medical information the system improves over conventional approaches and provides functionality that is unavailable in most conventional systems.

According to various embodiments, the application executed by a caregiver and/or the patient can provide options for defining a patient profile in establishing information on caregivers to be associated with the patient profile. For example, the user interface may display information on the patient, name, email, phone, cell, primary contact, among other options. The profile can include information on a specific procedure that the patient has undergone or is planning to undertake. Information on the patient's doctor and/or care team may also be part of the patient profile. According to some embodiments the patient may establish a list of caregivers who may assist with executing a care plan and/or be contacted with any issues to resolve.

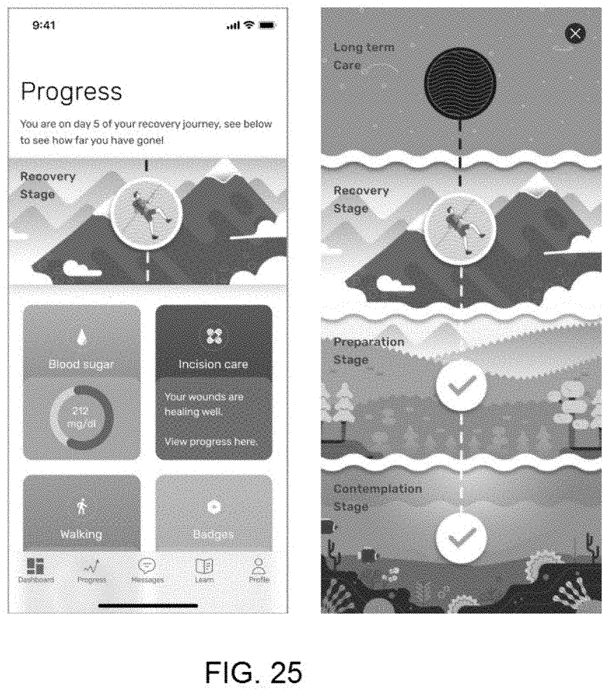

Shown in FIG. 25 are additional user interface screen captures. According to one embodiment, a holistic and continuous care plan can be shown to end-user. Various implementations of the management system improve over known approaches that simply provide a list of medical tasks. In various embodiments, the content engagement is tailored to the user and designed to be continuous through phases of contemplation to an end phase of recovery. The system may be designed to change perception of a care journey through visual and written cues that are tailored to respective users and in addition and/or alternative are tailored to evoke a specific emotional/psychological response from the users. According to some embodiments, the user interface is further tailored to limit the display of a number of tasks and information associated with them. Under some environments and executions, patients/users experienced task fatigue, various embodiments of the current management system are configured to limit and/or reduce task fatigue experienced by users.

According to some embodiments, the management system can display a progress page to summarize the status of a care plan execution and provide information on specific tasks in the care plan. For example, the user may see status indicators and readings associated with their current tasks (e.g., blood sugar, incision care, walking, badges). In further embodiments, the system is configured to display and organize care plan tasks and other functions according to phases of a patient's care. For example, the user interface can be organized into a contemplation stage for making a decision about a procedure, a preparation stage for preparing for a specific procedure, a recovery stage post procedure, and long term care stage for managing and implications of a procedure.

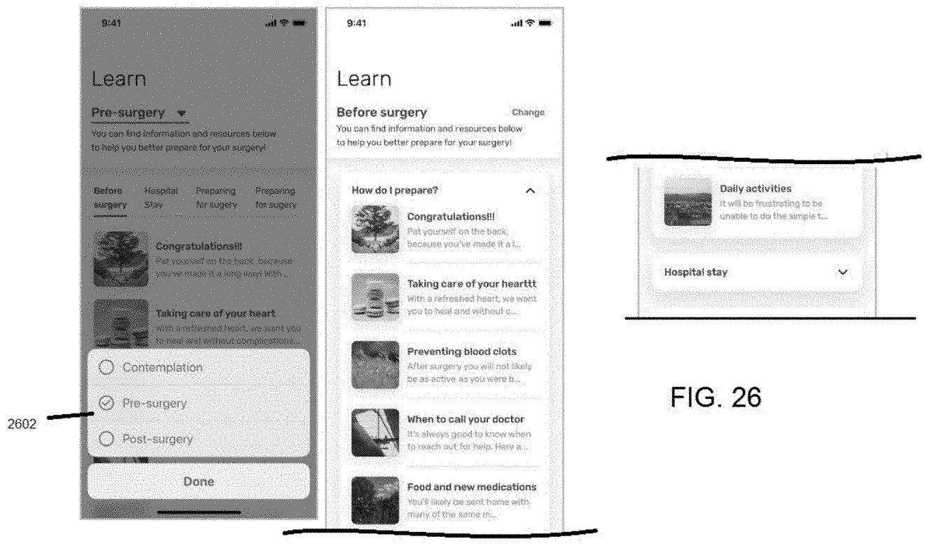

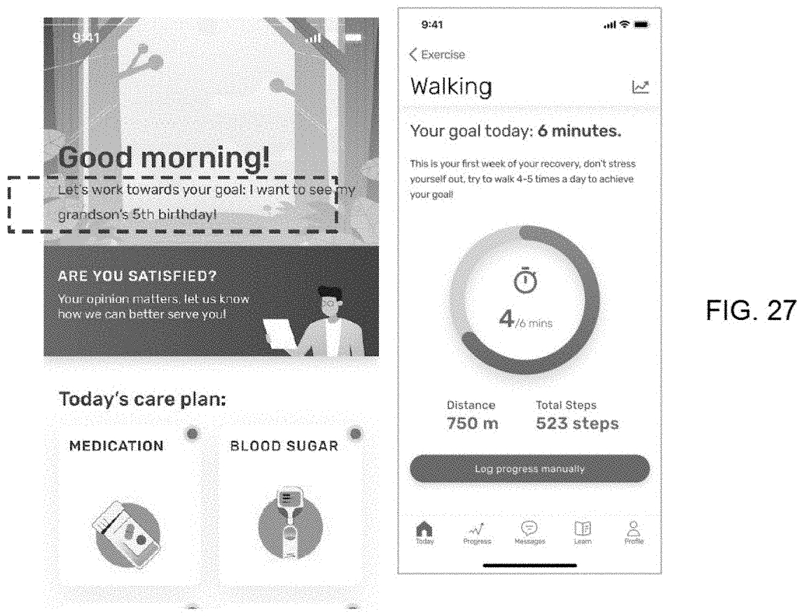

FIG. 26 illustrates further example screen captures from a user interface of a patient application associated with a management system, according to some embodiments. For example, in the contemplation phase the application emphasizes functions and operations to help a patient in making a decision around their care (e.g., to do or not in medical procedure). According to some embodiments, the user interface is specifically tailored to provide educational information regarding the contemplation phase, pre-surgery phase, and/or post-surgery phase. For example, the user may access tailored information based on a care phase with a single selection in the user interface (e.g., 2602). Responsive to selection in the user interface an ordered list of information sources can be presented in the user interface. For example, a patient may receive information on taking care of their heart, preventing blood clots, identifying when it you need to call your doctor, advice on nutrition, interaction between food and medication, and example daily activities that will improve the patient's outcome.

Shown in FIG. 27 are additional screen captures of example user interfaces. According to some embodiments the management system is configured to define a display personal motivational goals. The system implements user interfaces that are tailored to defining user goals and highlighting displayed information to enable completion of the same. In various conventional approaches in common failing is low activity compliance with medication, exercise, etc. Various embodiments of the management system resolve these issues via the defined goals, user interface highlighting, and emphasis among other options. In further embodiments, the management system establishes progressive goals and builds thresholds into each display so that even partial compliance with an activity is identified by the system and induces a response in the user interface (e.g., award of a badge, updated status indicator, we displayed progress meter, and dynamically alter display to reflect a new progressive section of an activity and/or progress).

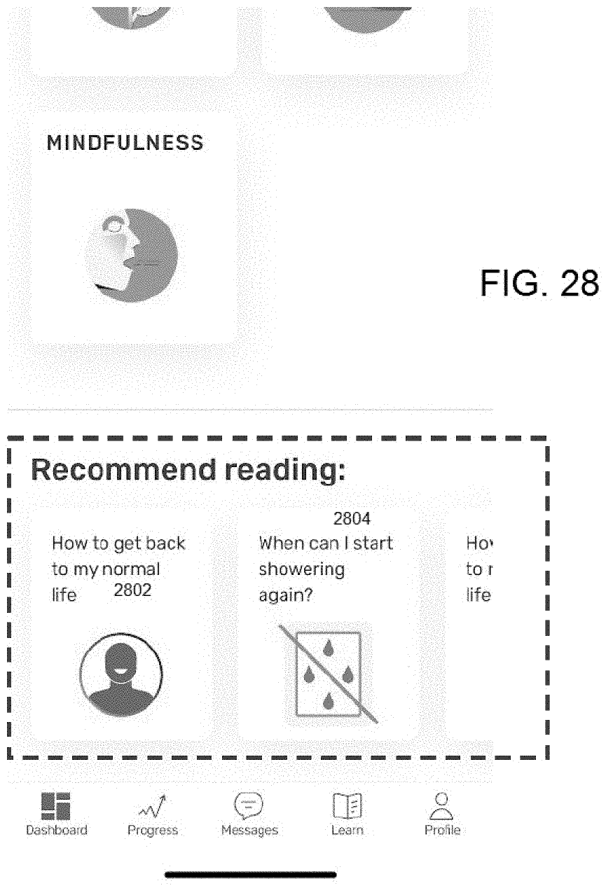

FIG. 28 illustrates a partial screen capture of a user interface. According to some embodiments, the user interface is configured to identify and highlight content for a patient that is tailored to their procedure and their profile, among other options. For example, the system can identify educational material that is particularly relevant to the patient's procedure and day-to-day tasks. For example, the system can identify questions asked by a large segment of similar patients. In one example, almost every patient will want information on when, and how, “to get back to my normal life” (e.g., 2802). In another example, the system can identify that a large segment of patients want to know the answer to the question “when can I start showering again?”. (e.g., 2804).

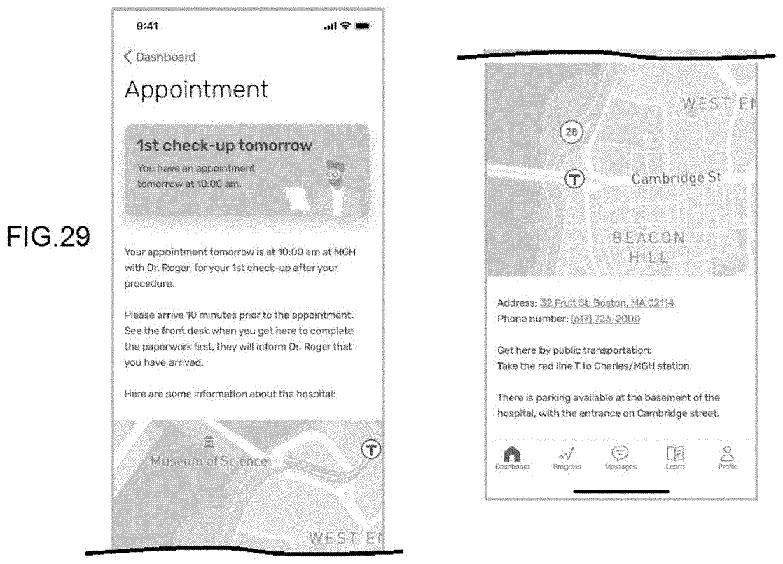

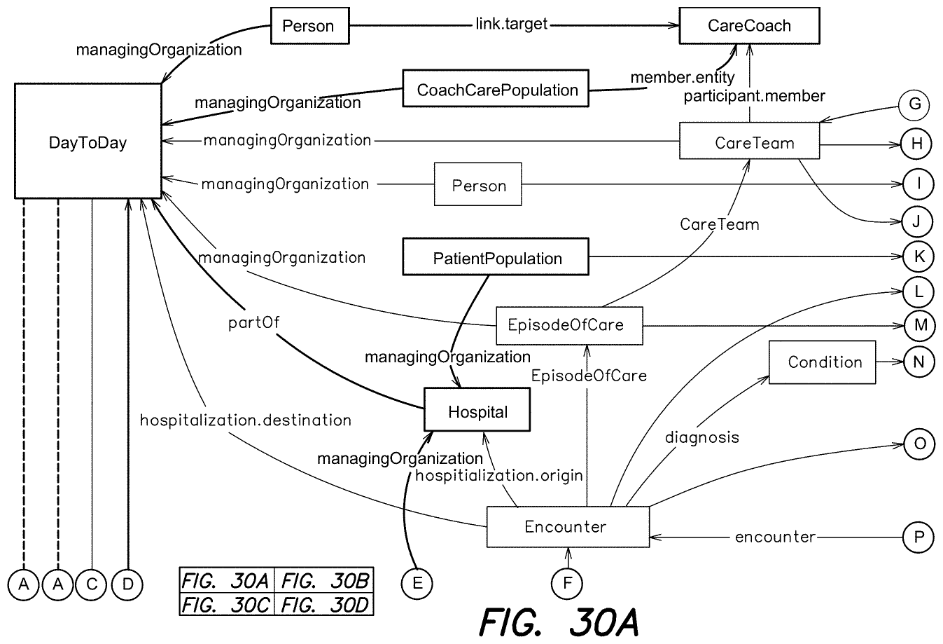

According to another embodiment, the management system is configured to collect and display a variety of associated information that simply is not provided in conventional systems. For example, FIG. 29 illustrates an example screen capture incorporating even driving directions to an appointment. According to some embodiments, the management system organizes such information into a task to be executed that can be displayed at a relevant time to the particular user. For example, the system can determine a ride time and/or traffic parameters associated with the trip and schedule the activity to begin at a time that will allow the patient to arrive for their procedure and perform any tasks once there.

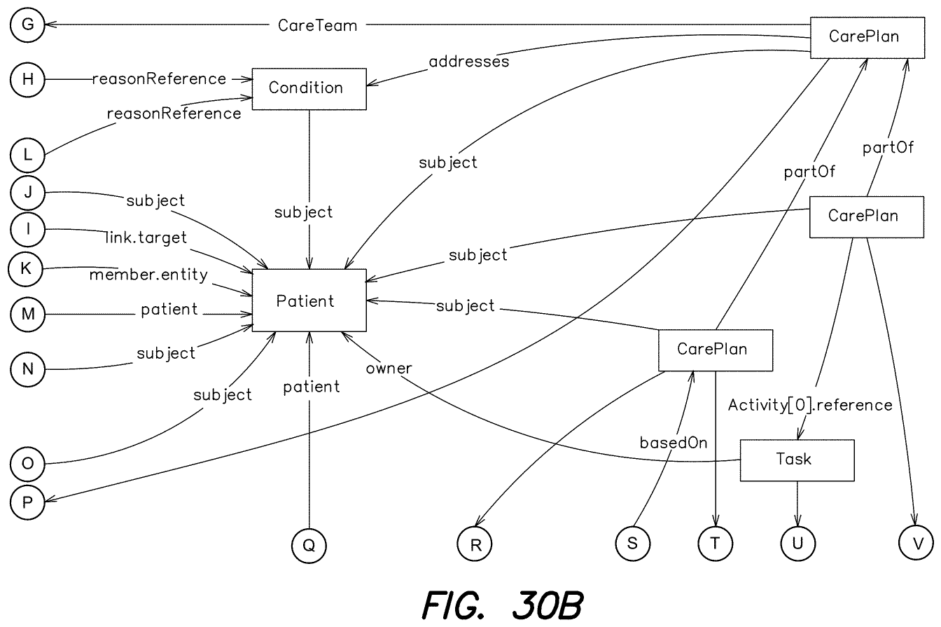

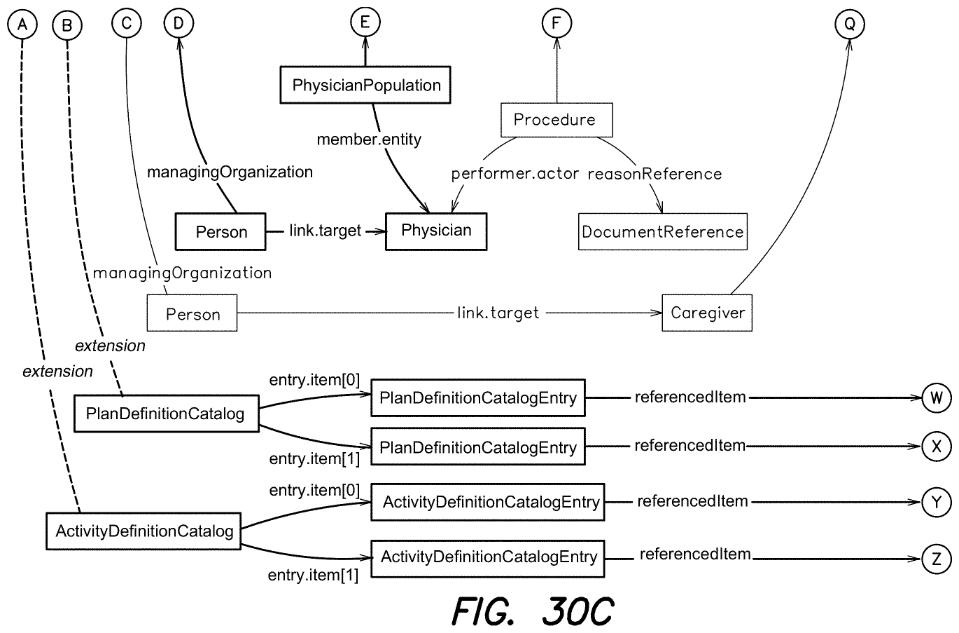

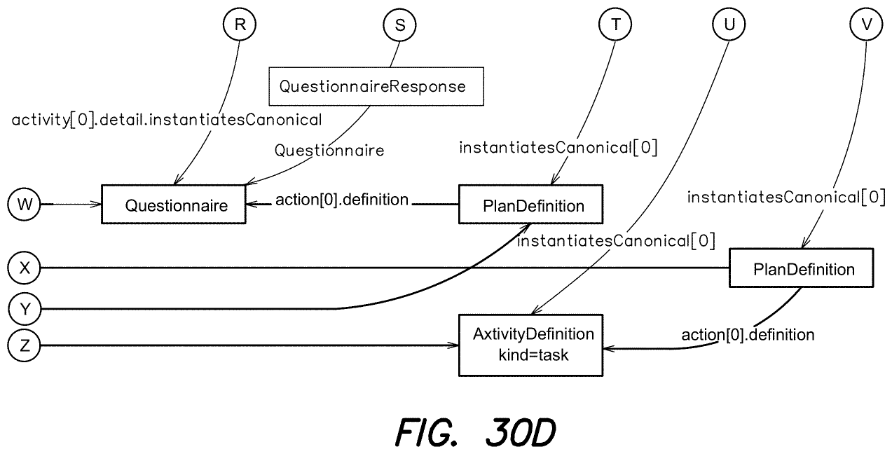

Shown in FIG. 30 is a sample object graph for care plan, according to one embodiment. As shown in FIG. 30 a respective patient can be a central spoke in the care plan mapping. Various care plans and/or steps in a care plan can be linked to a patient as a subject of the care plan task, step, and/or activity. In further examples, a patient may be linked to a condition as the subject of a specific condition that a care plan and/or care plan step addresses. Care plan, care plan steps and/or care plan actions can be linked to a specific care team which is linked to a patient condition by reason reference. Within each care team there may be care team members defined by a link (e.g. participant⋅member). For example a care coach can be defined as a member of a care team from a care coach population defined on the system (e.g. day to day block). In some examples the system may include definitions that specify members of the care team, care coach, care coach population are person objects that can be used to populate fields requiring person objects. For example a care coach population can be defined based on a group of qualified participants were labeled as person objects.

According to various embodiments, a patient may be identified from a patient population that is linked to a specific entity (e.g. hospital as a managing organization). In some examples, and encounter at the entity defines specific information for respective patients (e.g. a condition linked to a patient by a diagnosis reference). Additional information can be mapped in the care plan based on an encounter. For example a respective patient may be the subject of a specific encounter in and care plan, care plan step, care plan action can be the driving element in an encounter that takes place at an entity (e.g. hospital).

In various embodiments, entities that subscribe to the system can include a population of physicians and or other care personnel. The system can include mappings and/or definitions to physician population that can be assigned to an entity and/or selected as members of the care team. The various physicians in the definitions can be linked to an encounter via a procedure and a performer⋅actor reference. In some examples, the procedure will include a link to additional information describing why the procedure may be needed, and/or activities that may be required as a result of the procedure via a reason reference in the mapping.

According to some embodiments, the system provides access to a plan definition catalog. The plan definition catalog includes care plan steps, actions, activities, nutrition, and/or educational information that can be built into a care plan mapping. According to some examples, the plan definition catalog can include a plan definition catalog entry which is linked to a questionnaire by a reference item link, and the questionnaire can be referenced by a plan definition object. The plan definition objects can be integrated into a care plan to be executed by patient. In further embodiments, a plan definition catalog will include a plurality of plan definition catalog entries that can be selected and integrated into a care plan for patient. In yet other embodiments, care plan definition on the system may also include an activity definition catalog. For example the activity definition catalog may include a plurality of actions to be taken by a patient based on a specific condition, procedure, and/or a desired recovery outcome. According to one example, entries within the activity can include definition catalog specific entries in each entry may include a reference to a specific plan definition object. In one example the activity entry can be linked to a plan definition object which is also linked to a questionnaire to request information from a given user about execution of the activity specified in the entry. According to further embodiments, an activity definition catalog entry may also include an activity definition kind value which can be used to group or identify specific tasks.

Various embodiments, include a multitude of additional mappings to define a complete care plan. In further embodiments, the care plan mapping can include multiple conditions and multiple encounters each with respective mappings. In still other embodiments, the object graph for specific care plan may be adjusted during execution. For example, whether or not the patient performs specific tasks in the care plan may require adjustments to the specific plan being executed, and the object graph may be updated with new tasks new activities which are then saved so the care plan may be executed by the patient.

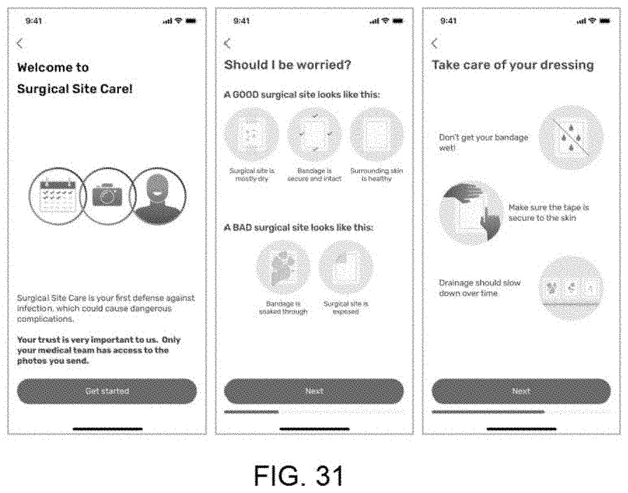

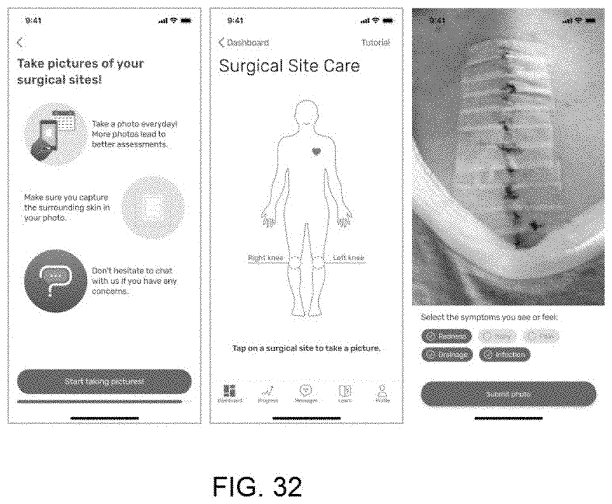

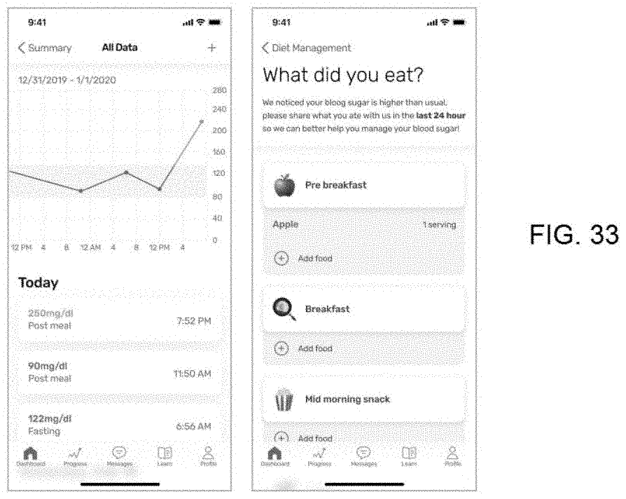

FIGS. 31 and 32 illustrate additional screen captures that educated a patient on their procedure and provide examples that enable the best self-care by a patient. An example is shown in FIGS. 31 and 32, in which the user interface displays variety of examples of surgical site characteristics, which can also include images associated with good and bad conditions for their procedure. FIG. 33 is another screen capture of a user interface that provides information to a user on their nutrition and/or blood sugar levels. According to some embodiments, the management system is configured to correlate nutrition inputs provided by a patient and blood sugar levels. The management system improves over conventional approaches as nutrition correlations can be developed in real time and recommendations on nutrition (e.g., different foods, different eating times, etc.) can be provided in real time and can also be provided based on predicting increases in blood sugar level.

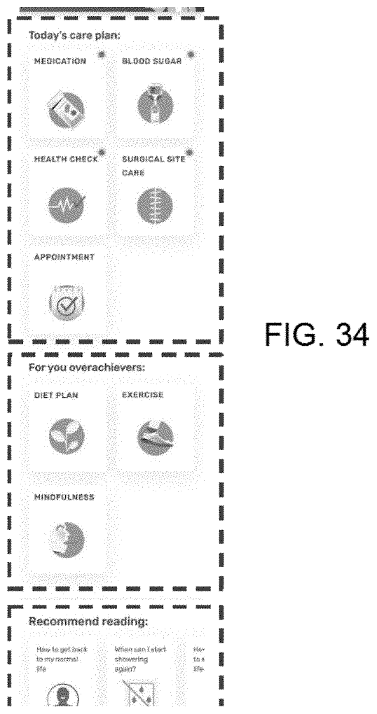

FIG. 34 illustrates an example user interface organized based on a visual information hierarchy. The inventors have realized that information overload remains a significant hurdle to conventional approaches. Various embodiments of the management system overcome the information overload problem based on the information hierarchy implemented in the user interface. For example, the system is configured to put the most important information at the upper portions of the display and relegate less important content to the lower sections—including, for example, tailoring a recommended reading section to a given patient and a specific day instead of against the full library of information. In various embodiments, by filtering the information based on day paradigm, the system can limit the information that needs to be analyzed in order to determine tailored recommendations for a specific patient. In addition, recommendations limited to a time window (e.g., day), prevent information overload and significantly improve compliance with medical tasks. Various embodiments implementing the information hierarchy provide significant improvement over conventional approaches.

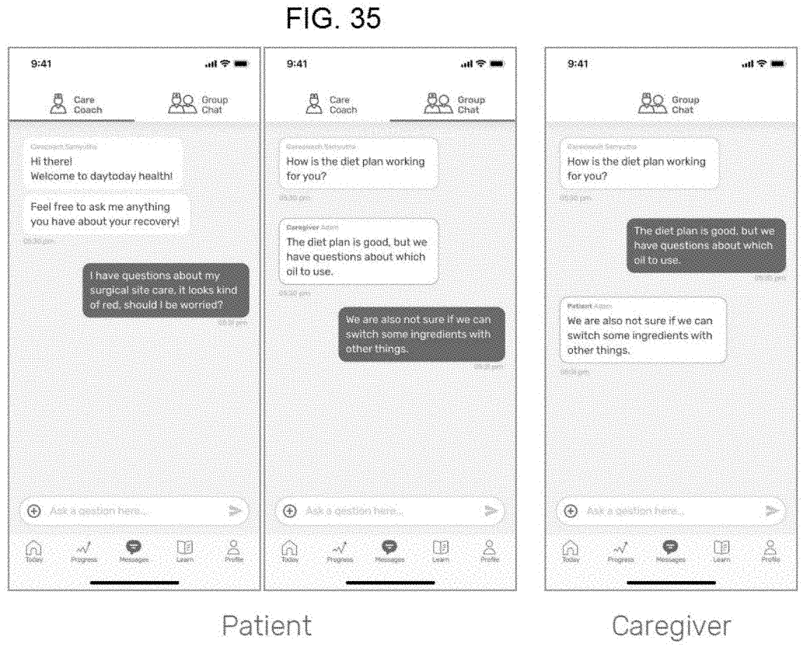

FIG. 35 illustrates example screen captures reflecting interaction between a patient and caregiver on the system. Various embodiments of the management system include a patient controlled separate chat channel that keeps caregivers in the loop and up to date on the patient's care. According to various embodiments, the patient controlled chat channel is configured for group chatting so that any identified caregivers in a patient profile can access and receive up-to-date information and information on actual conditions. According to some embodiments, the management system is configured to limit the communication channel to group chat settings such that no care provider can be excluded from the communication session.

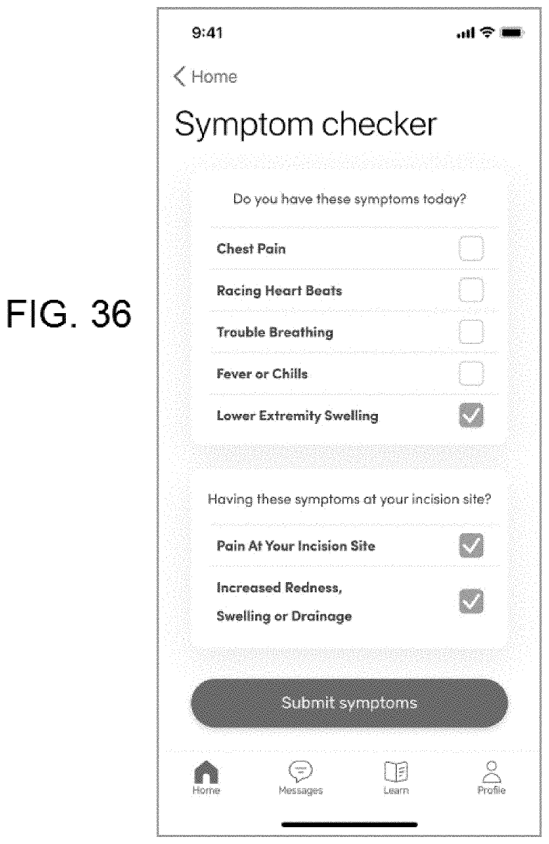

FIG. 36 illustrates another example screen capture that improves upon standard symptom monitoring systems. According to some embodiments, the management system provides a tailored symptom checking feature that displays to the patient a set of relevant symptoms or signs of complication based on their assigned care pathway, personal medical risks, and comorbidities. According to various embodiments, the system is configured to display the symptom checker more prominently during high-risk times and/or tailor the user interface to emphasize the symptom checker during time periods identified as having higher risk. In one example, the system is configured to emphasize the symptom checker functionality during times right after procedure and worn during the initial window of recovery.

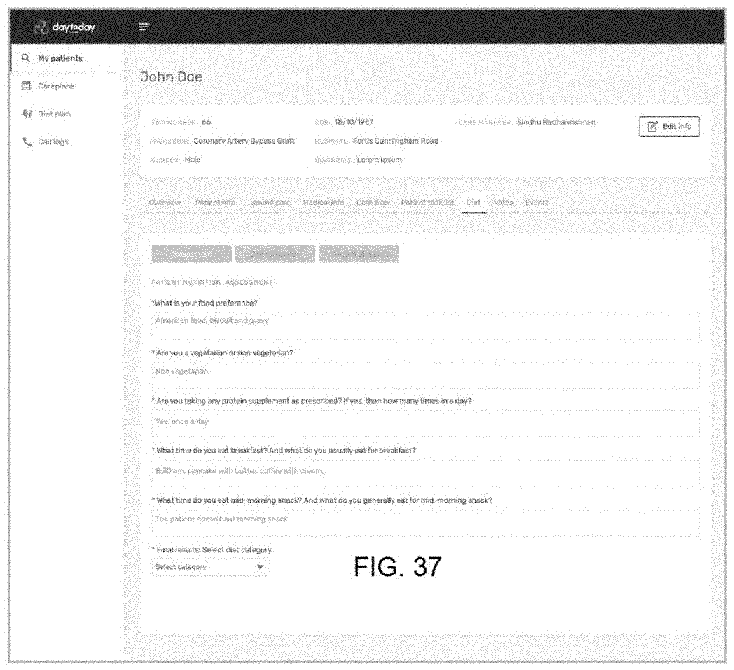

According to various embodiments the management system is configured to improve over conventional nutrition systems. For example, the management system is configured to analyze socioeconomic and cultural features in developing a dietary plan as part of a care plan executed by the system. Shown in FIG. 37 are example questions that can be asked of a user or patient to establish the various considerations for developing a meal plan. For example, the user can be asked what is their food preference, vegetarian or non-vegetarian, are you taking any protein supplements, prescribed nutrition, typical breakfast time, midmorning snack, timing, and any other information the patient wishes to provide.

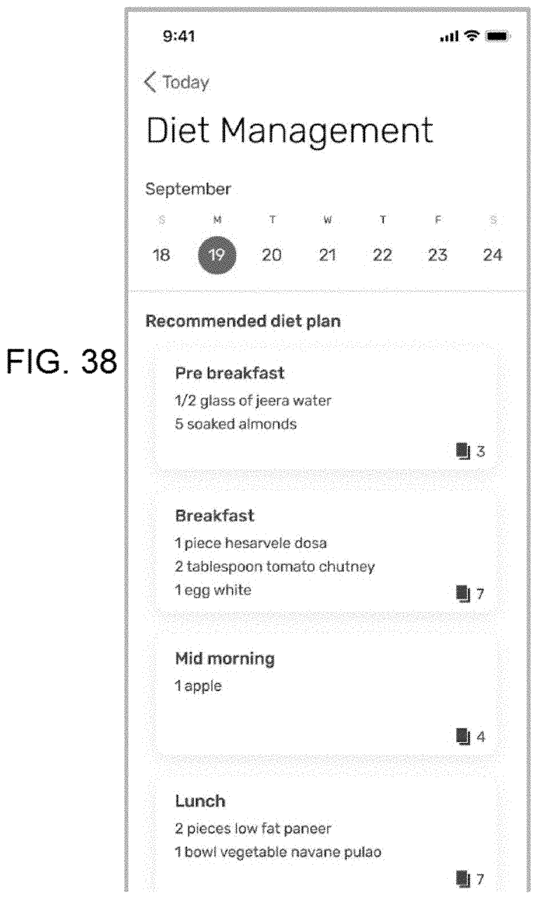

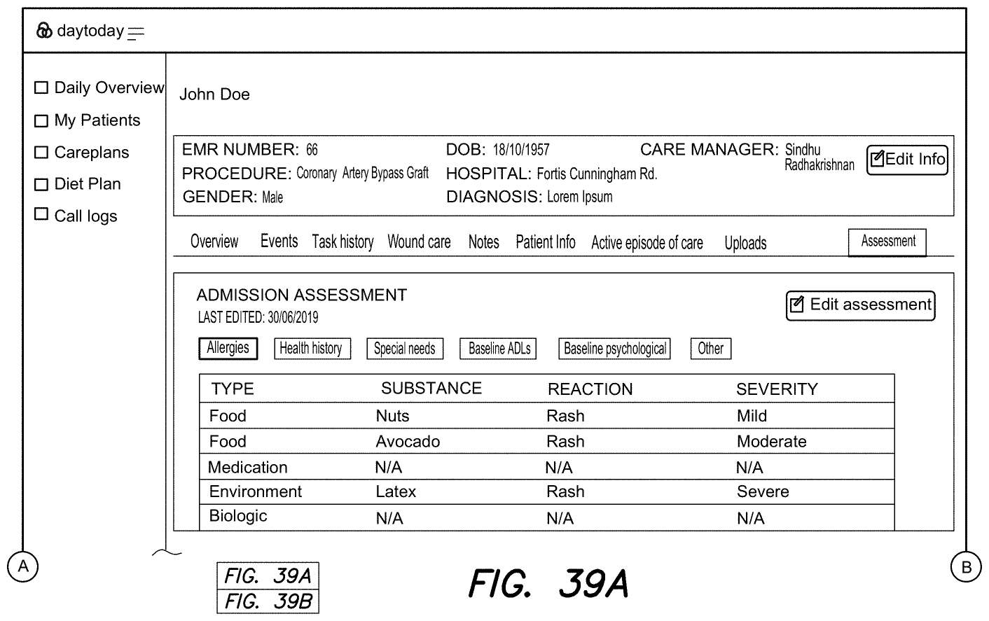

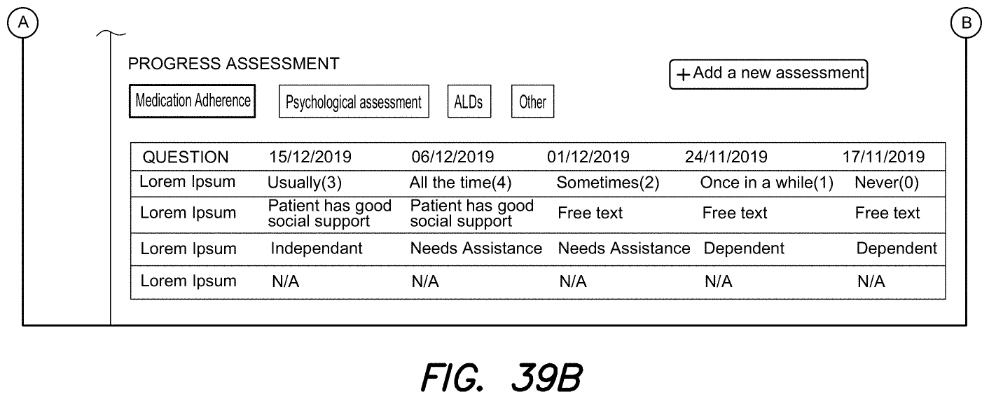

FIG. 38 illustrates an example diet management plan for given day. FIG. 39 shows an example screen capture of a care team dashboard that is configured to display an assessment of patient risk level based on a combination of surgical site monitoring, medication, blood sugar and diet among other options. According to some embodiments, the management system significantly improves over standard remote monitoring applications based on improved patient risk assessment and comprehensive holistic modeling of relevant clinical and personal factors—which can include the trends of the same metrics over time.

### Example Data Transformation

Various embodiments of the system are configured to handle multiple data format and differing data streams including health related data. For example, the system supports the acquisition of data from partner systems, such as HER systems (e.g. Epic, Cerner) and partner-provided files such as CDAs and CSVs, among other options. In addition, the system is configured to ingest and transform IoT originated data in raw and/or aggregate form.

Various embodiments employ domain specific language definitions to enable an editor for the FHIR Mapping Language (FML) and generate executable code that performs translations from provider-supplied source data to management system supported data. In various examples, the code is compiled into cloud based executables that can be run from any provider (e.g., can be run from a Kinesis Data Stream) between the provider and the management system. For each provider, there shall be a collection of data streams and associated FML documents—for example, one per message type. In further embodiments, code generated by the MPS FML editor can be defined in Java or Javascript, among other examples

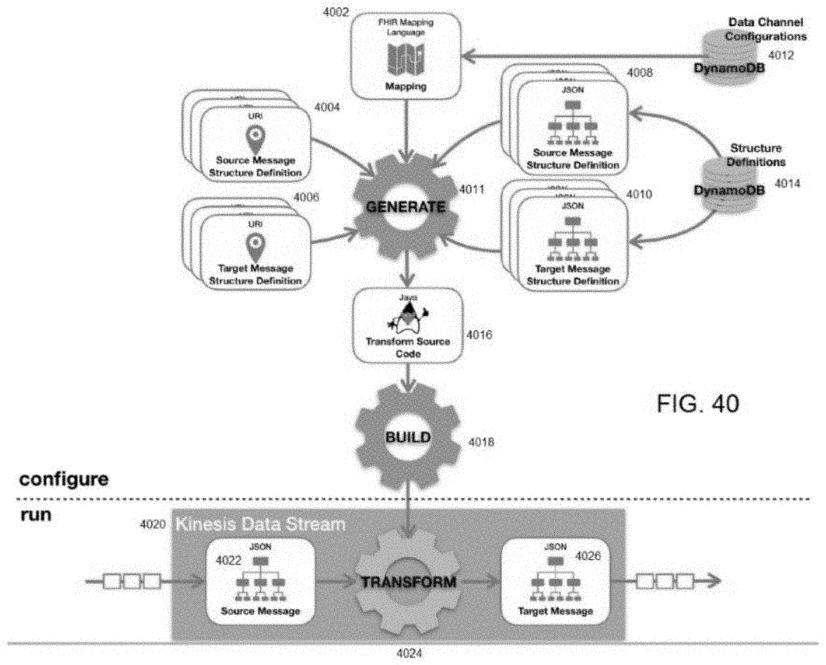

FIG. 40 shows an example process flow for data transformation. In the illustrated example, the diagram is partitioned into pre-runtime configuration steps (top) and runtime execution (bottom). According to various embodiments, the pre-runtime configuration steps are: generate 4011 and build 4018. In some embodiments, the generate step 4011 emits source code (e.g., 4016) that the build step 4018 compiles into an executable 4024 (e.g., a Lambda Transform) that can convert an incoming source message 4022 into an outgoing target message 4026, which can be executed as a data stream process (e.g., 4020).

Various embodiments are configured to generate code 4016 (e.g., Java code) that is configured to transform source data to target data. The system is configured to process the data types behind this data and to convert data from type into another, or possibly the same type. The data types can be unique to specific data channels, and each channel can have its own configurations (e.g., 4012).

According to one embodiment, the system employs structure definitions (e.g., 4008 -4010) to manage this functionality which can be stored in a database of structure definitions (e.g., 4014), and matched to a given data channel/configuration. For example, a FHIR StructureDefinition resource defines the data type for instances of entities such as a FHIR resource, HL7V2 message or IoT data packet. In another example, a data type is expressed as a StructureDefinition. According to another embodiment, the system is configured to transform data s of StructureDefinition S (e.g., 4004) to data t of StructureDefinition T (e.g., 4006). The can be configured with both a conceptual mapping between the elements of S and T as well as content transformations from s to t. In some

In various embodiments, both conceptual and content mappings are captured in a FHIR StructureMap resource. This can be expressed by the system as either JSON or FHIR Mapping Language (FML) artifacts (e.g., 4002). A desired output can include a StructureMap with FML that can be employed to generate code that can transform data s of type S to data t of type T. In some examples, the output code is referred to as a solution.

In various embodiments, the data messages that the system is configured to ingest sourced from a provider come in N different types. Thus, the management system can be configured the N different types multiplied by the number of providers to deliver a total number of solutions that are supported.

In various embodiments, domain specific definitions and transformations provide solutions compliance with FML. In various examples, an Abstract Syntax Tree (AST) that results forms the basis for generating Java source code that transforms source data into target data. For example, the generated source code can be built into an AWS Lambda for use in a Kinesis Data Stream.

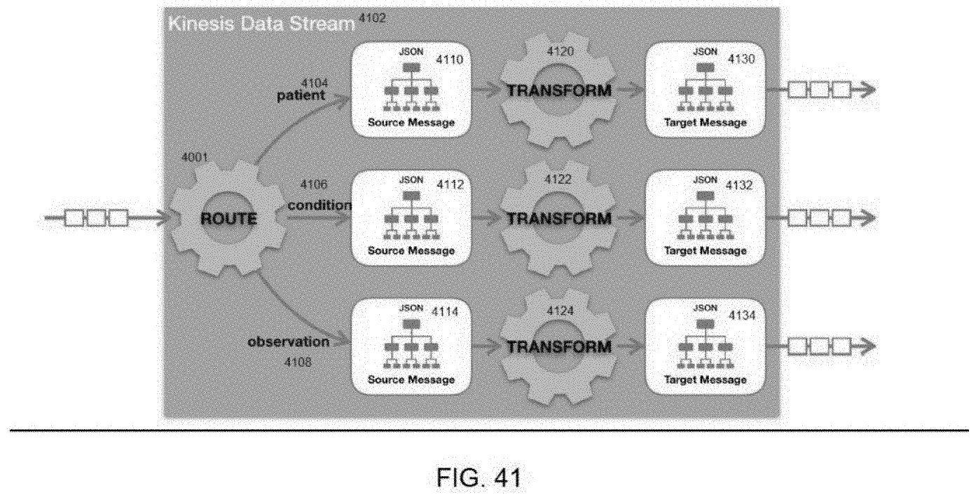

FIG. 41 illustrates an example embodiment and an example data stream (e.g., 4102) for each data source (which can include, for example, a hospital). In various embodiments, each stream is subdivided into separate processing paths for each supported data type (e.g., 4104, 4106, 4008) by on a router process (e.g., 4101). For example, hospital H would have data stream hi which is comprised of separate processing paths for each supported FHIR resource type (e.g. patient 4104, condition 4106, observation 4108).

In various embodiments, an AWS Gateway API endpoint is architected to manage communication between a provider and a respective Kinesis Data Stream. In one example, when the endpoint receives a message-transformation request, the data type is extracted from the payload and the message body (e.g., 4110-4112) is routed to the appropriate data processing path (e.g., 4104-4108), where the transform (e.g., 4120-4124) converts it to a target message (e.g., 4130-4134).

In some embodiments, the target message is a FHIR Message resource that contains a bundle of other FHIR resources. For example, a FHIR Patient resource received from a provider would be transformed into a FHIR Message intended for the management system. This FHIR Message can contain a FHIR Bundle resource which would itself can contain other FHIR resources such as Person and Patient. The management system can be configured with various endpoints that accepts FHIR Messages with this structure.

### Example Implementation and Architecture

According to various implementations, a management system includes architecture and system functionality to provide:
optimized standard procedure care journeys—care plans that cover not just what each hospital already does for each procedure, but include the evidence-based optimizations/standards in holistic care for patient outcomes, and are iteratively updated and optimized for different patient segments, cultures, etc. In various embodiments, the system tailors presentations of the care journeys as individual steps to take on a daily basis, wherein the displays provide reminders for each individual steps, and can also include confirmation or reminders of execution for each step of a particular day or days. In some examples, the system analyzes compliance issues with steps of a care plan and adjust the execution, display options, timing, and/and reminders to improve compliance scores. Various embodiments, incorporate historic analysis and pattern matching to update care plans and/or daily activity steps to improve compliance with execution.
holistic procedure remote coaching—chat and video coaching that is not just open ended, but that the system structures to follow continuously improving care plans, which includes all aspects of optimal preparation and recovery/rehabilitation from procedures (e.g., hysiotherapy/exercise, nutrition modification, stress and sleep management, caregiver training, etc.) that the system provides and monitors for execution. In some examples, the system analyzes specific execution information for the individual patient and tailors the step-by-step execution plan as compliance information is obtained by the system. In further embodiments, the system can be configured to automatically initial coaching session for tasks having low compliance scores, and/or compliance scores that have a worsening trend. In various embodiments, the system can analyze prior execution and/or deviations in care plan execution to identify options for improving compliance scores. In one example, the system is configured to dynamically re-order user interface displays for a patient to provide emphasis and/or highlight activities on which a user is scoring poorly (e.g., below a threshold compliance). In further example, historic analysis can demonstrate higher compliance scores on activities scheduled early in the morning. The system can reschedule activities with lower scores to occur in the optimal time window identified. In further embodiments, the system automatically rotate the timing of executions of various task to ensure a baseline compliance score is maintain across groups of tasks. In various embodiments, timing and scheduling can automatically be adjusted by the system. In additional embodiments, the system can track other user activities and develop inferential information that affects compliances score. For example, physical rehabilitation session may negatively impact compliance score for other activities for a given patient, and responsive to such analysis, the system can re-schedule daily activities to avoid negative effects.
holistic patient education—content that is not just medically accurate and minimally adequate, but is customized by the system to be engaging (e.g., experts establish various optimization and/or options that the system can dynamically select for a given patient, care journey, etc. and further that the system can modify and update during an execution, among other options). In various embodiments, the system is configured to tailor content into a step by step process that is interactive, and covers medical information and instructions (including, for example, nutrition, stress/psychosocial/sleep data collection, physiotherapy options and execution, as well as caregiver support, among other options).
interactive engagement architecture—for example the system includes downloadable applications and tools presented on a patient's computer or mobile device. These applications and tools are configured for more than content display, and present game based treatment execution, learning algorithms, and behavior theory analytics to drive sustained engagement (e.g., update customized displays and/or care plans) with content and create a sense of narrative and progress arc for patients.
patient management dashboard: various embodiments of the system tie patient care directly to the care team managing their health. The system can integrate with remote health monitoring devices at patient locations, collect, and analyze captured data(e.g., blood pressure, activity, heart rate, respiration exercise, EEG data, among other options) to provide granular patient care tracking. In one example, a care team dashboard is generated and displayed by the system, that enables a care team to monitor patients and capture and present at high resolution key clinical data, as well as patient reported data and outcomes, and can also include clinical notes for improving the learning models supporting the system, functionality, and architecture. In one example, responsive to care team feedback, the system can dynamically redefine a care journey or optimize the care journey for the specific patient. In further examples, learning algorithms can be updated for subsequent execution as various care journeys are used. Ultimately various system embodiments capture, analyze, and present health data faster than conventional approaches, enabling complication prevention and enabling efficient care team interventions at scale (e.g., which cannot be done under various conventional approaches).

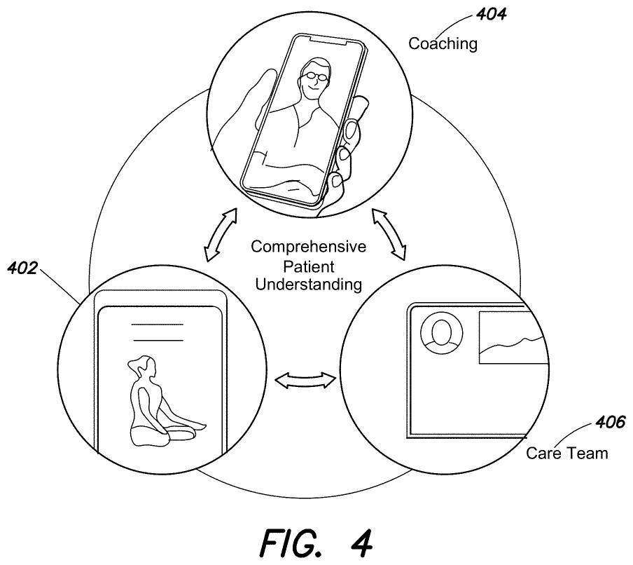

FIG. 4 is a conceptual illustration of elements of a management system and the engagement that the system fosters between patients, educational and care content 402 that is tailored to the user's needs in an manner that any user can actually use and understand, and a care team 406 that supports the patients/users. Various embodiments provide coaching services that facilitate patient compliance with their care plan, and enable engagement between the patient and their care team (including, for example, their physician, nurses, therapists, nutritionists, etc.). In addition, the system hosts and can automatically trigger live coaching services 404. For example, the system can detect reduction in compliance scorings for various care plan activities or with a phase of a care plan. In some examples, responsive to detecting a reduction in compliance with a care plan, the system can trigger live coaching sessions at care plan activity events. In various embodiments, the coaching can facilitate patient compliance and resolve issues that the patient has not reported by automatically triggering such coaching intervention.

In further aspects, the system provides unique perspective to the care team managing a patient's recovery. For example, the system is configured to generate and display a care team dashboard that captures and displays at a high resolution key clinical data, patient reported data and outcomes, and clinical notes. The various aggregations and data sources that system collects can be used to train machine learning models that are then used to augment care plans. For example, care plans and outcomes for respective conditions, procedures, etc., as well as contextual information for the patients following the plans can be used as training inputs to a machine learning model. Trends reflective of improving compliance scores can be identified by the model and used to suggest and/or selection options for improving care plan. Execution information can also be used to train machine learning models, such that the system can identify trends and/or issues in respective patient execution of a care plan. The system can be configured to adjust the activities, scheduling, introduce coaching, etc., where the machine learning models indicate a likelihood of improving patient outcomes/compliance.

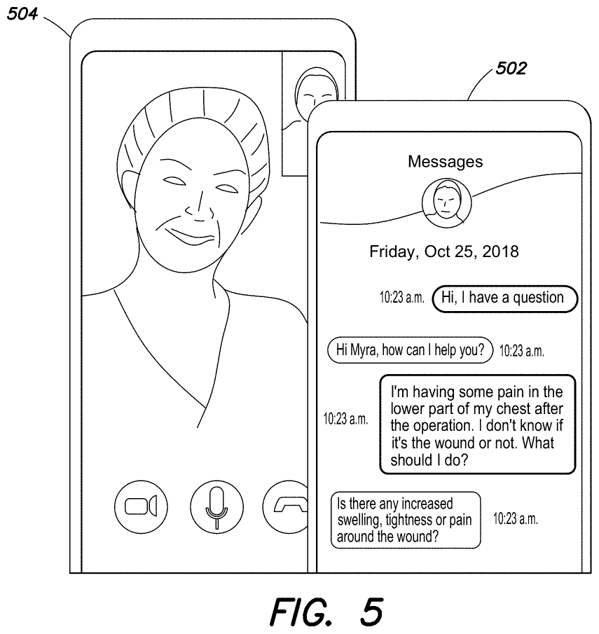

FIG. 5 illustrates functions for building engagement between the care team through coaching options and the user/patient following the care plan/ In various embodiments, the system provides functions for easily accessing a care team member to answer questions, address issues, before they become a source of re-admission risk. For example, a patient/user can access text messaging services (e.g., at 502) to ask questions, submit images associated with their issues, and receive educational support (e.g., example images of their care issue (e.g., for wound care images of a typical surgical site at a patient relevant time)) within an intuitive user interface. Additional the user can trigger additional communication as needed (e.g., at 504) to have live video interaction with a care provider or care team member.

In some embodiments, the interaction between patient and care team can be initiated by the user. In other embodiments, tracking on the system of patient information can trigger automatic execution of the coaching services. For example, as care plan events are completed or scheduled to be completed, the system can automatically trigger coaching check ins between the care team and a patient.

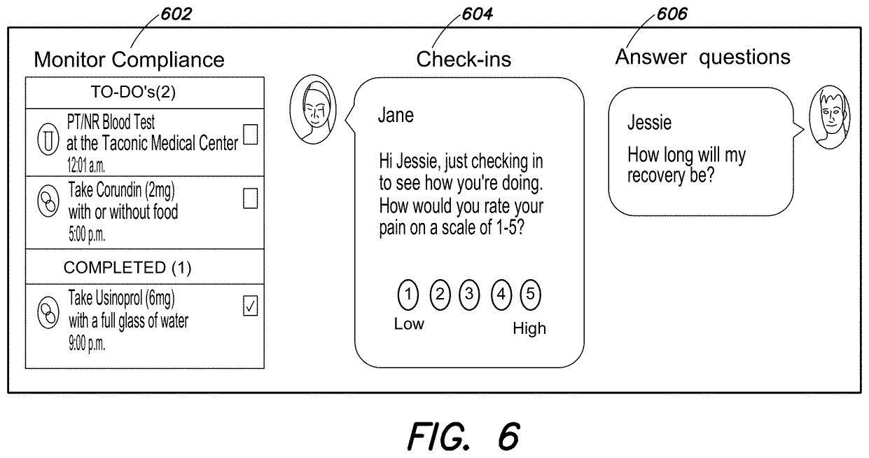

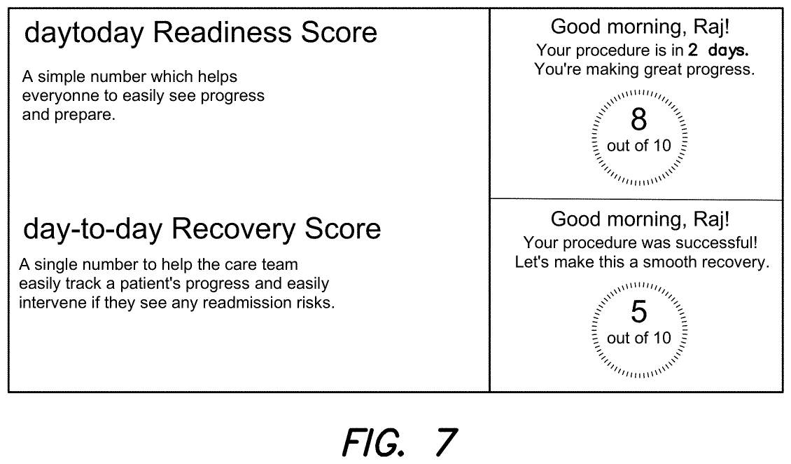

FIG. 6 illustrates examples of interactions between the care team and patient user. For example, the care team members can access and monitor daily compliance as well as historical compliance with a care plan at a glance (e.g., at 602). In further embodiments, the system triggers automatic check-ins between the care team members and respective patients (e.g., at 604). The system an prompt such automatic check-in based on analysis of compliance and/or compliance scores, pattern matching on care issues, on a schedule, periodically, among other options. The interface is also configured to enable users to ask any questions and have a member of the care team respond with little or no wait (e.g. 606). In various embodiments, the system seamlessly handles connecting the end user patient to a care team member by accessing a communication interface. The communication interface, which can include a mobile text display, is configured to handle connections to any member of the care team. For example, the system can identify the members of the care team, determine availability, manage a connection to the care team member and render the interaction so that the end users observes the entry of a question into a text box, and a corresponding response.

In further embodiments, the system can further facilitate patient understanding of their recovery by reducing compliance information and care plan execution information into simply compliance scores that are associated and displayed with daily execution tasks. For example, the system can generated and display a “readiness” score to respective patient based on tracking their compliance with an individual care plan. In another example, the patient action items and/or daily plan activities can be displayed with a “recovery” score. FIG. 7 illustrates an example display of readiness and recovery scores. Various embodiments tailor respective scores to the context of the care plan in which they are display. In some examples, the context can include a phase of care plan execution (e.g., contemplation phase, preparation phase, recovery phase, long term care phase, etc.). For patient, the system is configured to reduce the volume of information on care plan execution to a simple score display, that a patient can readily understand and adjust behavior to improve. Likewise, a care team can instantly access a patient and care plan execution based on the a displayed score. In various embodiments, the system is configured to identify re-admission risks based on trends and/or patterns in compliance scores.

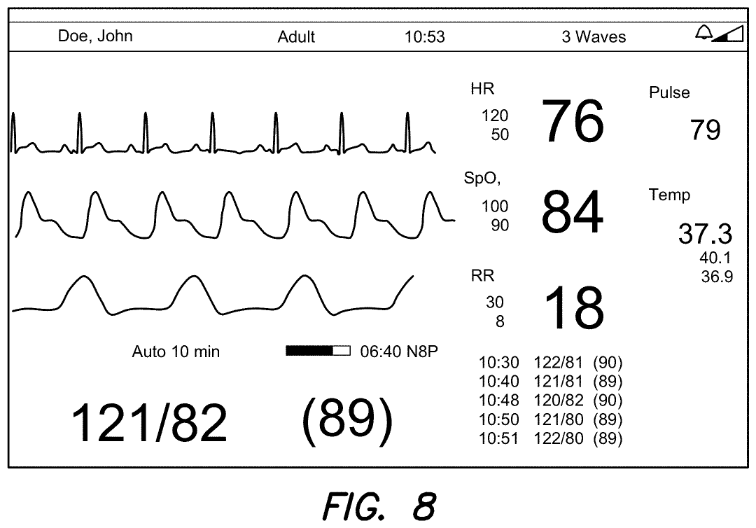

In addition to identification of re-admission risk based on the patient's activity and/or compliance score associated with a care plan, the system is configured to provide remote monitoring of patient health information. For example, FIG. 8 illustrates a remote monitoring display for heart rate sensors, blood pressure sensor, oxygenation sensor, breathe sensors, temperature sensors, etc. Various monitoring system can be associated with respective patients and used as part of monitoring discharged patients. Additionally, the system can integrate various user devices (e.g., heart rate monitors, activity monitors, step counters, etc.) and incorporate sensor data into the patient's compliance record. Various displays are configured to provide real time visualization of integrated sensors systems, including, for example, APPLE watch sensors, health monitoring devices, respiration monitors, EEG devices, etc.

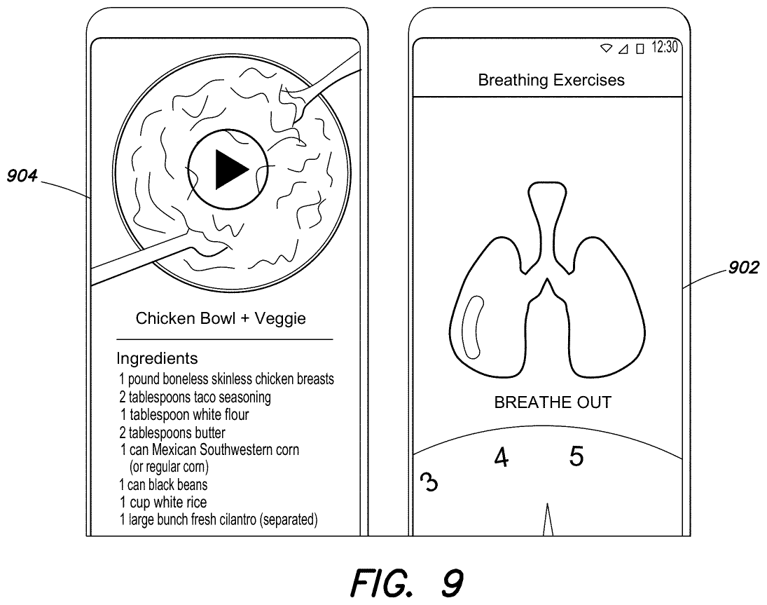

Further, the system defines a gold standard of patient engagement which includes, for example, tailored user interfaces configured to present relevant educational information and action interfaces. In some embodiments, the user interface is configured to limit the number of selection required by a user to transition from their care plan to day by day activities to single or a few selections in the user interface (e.g., 1, 2, 3, etc. selection). Each action item in a care plan is organized in the user interface to show what activities need to be performed on given calendar day. These action items can include preparatory actions (e.g., pack clothes, consume fluids, fast, rest, plan route and return (e.g., the system may automatically generated and integration driving directions into the action item, such that a singled click on an appointment time transitions the user interface to driving direction to an appointment or lab test—in some examples, for users having a “no car” setting, public transport routes or an ride service interface can be integrated into the display). The action items can include pre- or post-operative events, including, for example, take medication at scheduled intervals. In further example, an action item can specify “breathing exercise” to measure patient recovery. FIG. 9 illustrates an example user interface that guides a user through a set of breathing exercises and that records the patients compliance with the exercise as well as any results (e.g., 902). In further the user interface is configured to provides access to educational information relevant to the patient care plan. The patient tailored/care plan tailored content can include displays for nutrition options that are best aligned with the patient care plan (e.g., 904). Nutrition options for display can be selected based on a given procedure and timing of how far along a patient's recovery or care plan is. According to some embodiments, the patient tailored content is integrated into the care plan and/or action item displays such that the content is accessible, in some embodiments, in as few as one section or click.

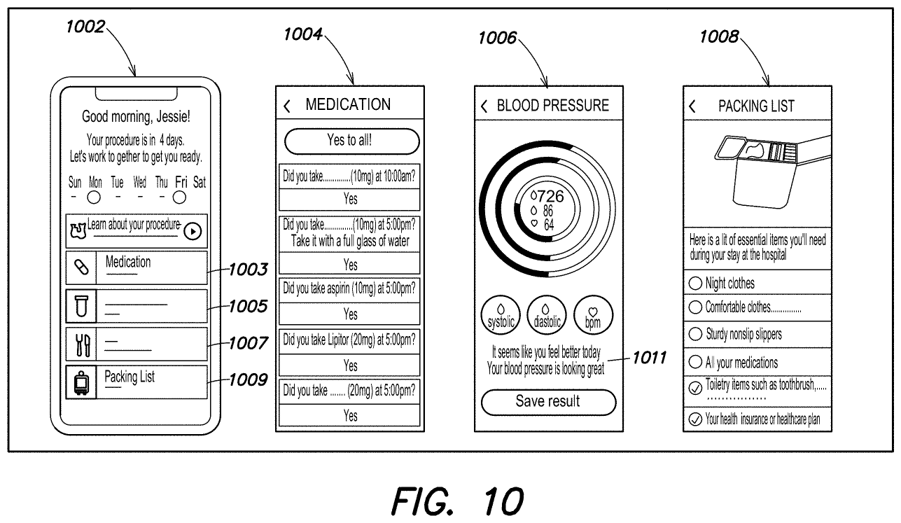

Illustrated in FIG. 10 are example screen captures of a care plan display organized by activity for a given day. At 1002 show is a screen capture of a care plan day of activity. The display is organized within the user interface based on action items that cover needed actions for a given day. Each action item display can be associated with a lower level display that provides more detail once selected. For example, a lower level display for medication can be displayed responsive to selection of 1003 “medication.” The display of medication for a given day (e.g., 1004) can provide a list of all medications and timing. In some embodiments, an application executing on a mobile device can be configured to trigger reminders and/or alarms based on the timing of medication described in a care plan. In some example, the application can automatically start at schedule times and bring the user directly the “medication” lower level display to input activity information. The system and/or application can be configured to reminder users of each and every activity in a care plan to ensure the actions are completed. Selection of pre-operative testing (e.g., 1005) can transition the user interface to a series of test s that user can perform before their trip to the procedure. For example, blood pressure sensors can be activated and data captures as part of a pre-op testing suite. Screen 1006 shows an example interface for displays information on blood pressure, and that include subtle education message on the state of their own health. The unique organization of the user interface further resolve issues associated with conventional hospital/patient operation. In once example, a packing list 1009 can include all medications, but also other needed item items. In another example, include “current medication” activity items into displays for a patient has improved patient readiness over conventional approaches. A common failing known in the conventional treatment settings is the failure of patient undergoing procedures to bring existing medications, and/or notify staff conducting a medical procedure of existing medicine. The example screen capture 1008 illustrates how the organization of the user interface itself, limits the requirement to search and identify information by a patient needed for a given procedure. Further screen 1008 illustrates a hierarchical organization of actions and needed items to fulfill a given action or set of actions. For any given action, the system is configured to generate alert and automated interventions. For example, the system can trigger lock out screens, alert messages, push notification, etc. to ensure/require a patient comply with actions specified in their plan. In further embodiments, the system can infer based on timing and/or location based information (e.g., movement, etc.) that a patient is preparing to go to an appointment and/or procedure. If any requirement remain unmet, the system can trigger automated phone calls to the patient as reminders and/or persons associated with the patient on system (e.g., spouse, family member, etc.). In one example, the user interface can include a selection for diet 1007 to provide recommendation on nutrition, meals, etc. that a patient should use to improve recovery. The diet/meal plan can include fasting periods for medication among other options.

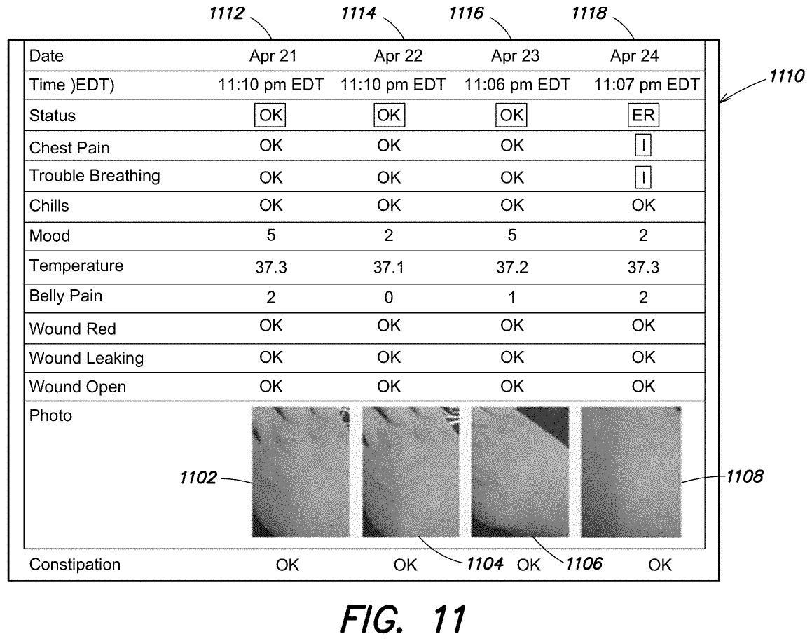

Various embodiments of the system provide a level of engagement that is unachievable in conventional settings and approaches. FIG. 11 illustrates an example interactive engagement screen 1100. According to one embodiment, various tailored user interface screens are made available on the system based on a patient procedure and/or post procedure care plan. Screen 1110 illustrates an interface for daily wound monitoring that enables early identification of issues, and can trigger preventive actions prior to conventional approaches. According to various embodiments, the system is configured to manage post operation care lifecycle. In this example, the system manages a wound care lifecycle. According to some embodiments, a patient care plan can include an action item to complete a survey and capture a photo of a wound site undergoing treatment. For example, the images at 1102-1108 show daily captures taken in compliance with care plan action items. In another example, survey responses shown at 1112-1118 show patient responses to questions presented as part of a care plan action item. The resulting information can be made available to a patient care team, and the system can identify potential issues to the care team and trigger automatic communications for the care team's review. For example at 1118, the system identifies and flags responses indicating an increase in chest pain and/or an increase in trouble breathing by the patient. The status shown in 1118 indicates “ER” as an indicator of potential need for immediate action. According to some embodiments, the system is configured to identify any changes in user responses that are indicative of a worsening condition and automatically communicate such changes to patient care team. In some examples, the system can automatically triggers a communication session between a patient and care team responsive to patient responses to condition. In yet other examples, the system can be configured to automatically trigger a video communication session to a care team member based on patient responses.

Various embodiments of the system are configured to make the ongoing care tasks for a patient post procedure effortless. According to some embodiments, the system enables autonomous care by the patient that integrates a responsive care team. For example the system provides learning guidelines and standardized procedure care plans that produce optimal care plans across patient segments. In further example, the system enables greater insights into respective patients where a whole person view is provided and communicated to a care team including, for example, psychosocial, cultural, and behavioral elements, among other options. In other aspects, the system facilitates communication between the patient and their care team and delivers contextual-based instruction to best enable self-care by any patient. Further, the system can also provide motivation to engage in and/or comply with the care plan. Encouragements can include automatic reminders, automatically triggered coaching sessions, automated phone reminders, communication to designated parties (e.g. family members, spouse, etc.), among other options. As the system captures information on care plan execution the system can be configured to improve machine learning models that recognize patient behavior patterns and/or predict issues with patient care in time to remediate.

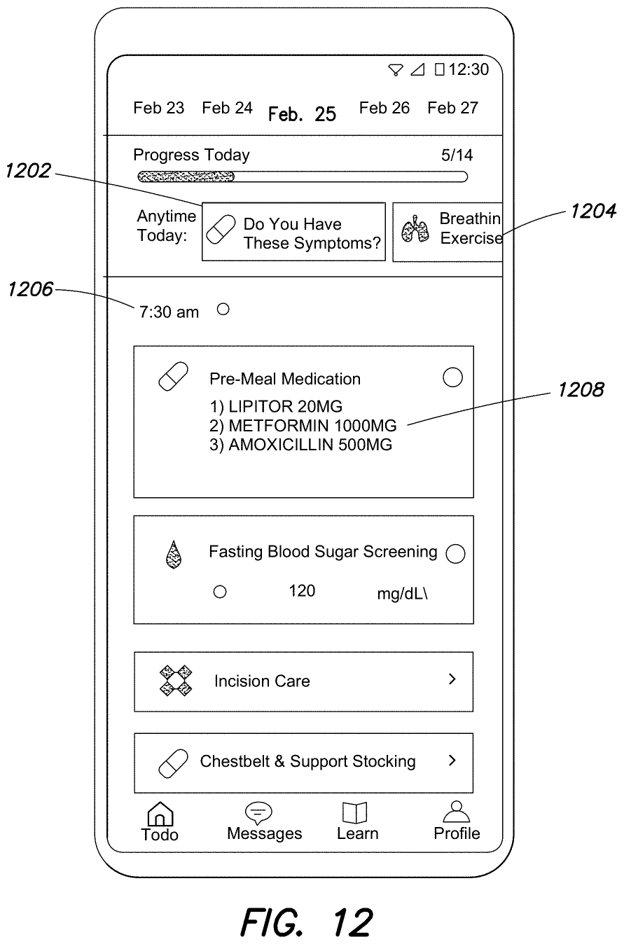

FIG. 12 illustrates an example screen capture of an interface showing daily progress rate for task completion. In this example, the system provides easy access to a symptom checker 1202 and contextual access to breathing exercise instructions 1204. Additionally, the user interface can provide a timeline to guide users through a daily schedule of tasks at 1206. At 1208, the user interface can provide information on the specific tasks and how best to complete them (e.g. “pre-meal”). The display can include information on medication types and dosing—and may include images of the respective medications available upon selection in the interface at 1208. In further embodiments, the user interface is configured to accept user input for data tracking within the timeline view to eliminate the need to transition between a variety of screens in order to capture information on a patient's activity.

### Example Computer Implementations

Various embodiments discussed above may be implemented to improve the function of one or more computer systems. These computer systems may be, for example, general-purpose computers such as those based on Intel PENTIUM-type processor, Motorola PowerPC, Sun UltraSPARC, Hewlett-Packard PA-RISC processors, ARM Cortex processor, Qualcomm Scorpion processor, or any other type of processor. It should be appreciated that one or more of any type computer system may be used (and thereby improved) to partially or fully automate the functions, features, and/or operation described according to various embodiments of the invention. Further, the system may be located on a single computer or may be distributed among a plurality of computers attached by a communications network.

The computer system may include specially-programmed, special-purpose hardware, for example, an application-specific integrated circuit (ASIC). Aspects of the invention may be implemented in software, hardware, or firmware, or any combination thereof. Further, such methods, acts, systems, system elements, and components thereof may be implemented as part of the computer system described above or as an independent component.

The computer system may be a general-purpose computer system that is programmable using a high-level computer programming language. The computer system may be also implemented using specially programmed, special purpose hardware. In the computer system there may be a commercially available processor such as the well-known Pentium, Core, Core Vpro, Xeon, or Itanium class processors available from the Intel Corporation. Many other processors are available. Such a processor usually executes an operating system which may be, for example, the Windows NT, Windows 2000 (Windows ME), Windows XP, Windows Vista or Windows 7, 8, or operating systems available from the Microsoft Corporation, MAC OS X Snow Leopard, MAC OS X Lion operating systems available from Apple Computer, the Solaris Operating System available from Sun Microsystems, iOS Blackberry OS, Windows 7 Mobile or Android OS operating system or UNIX available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

As discussed, one or more portions of the computer system may be distributed across one or more computer systems coupled to a communications network. These computer systems also may be general-purpose computer systems. For example, various aspects of the invention may be distributed among one or more computer systems configured to provide a service(e.g., servers) to one or more client computers, or to perform an overall task as part of a distributed system. For example, various aspects of the invention may be performed on a client-server system that include components distributed among one or more server systems that perform various functions according to various embodiments of the invention. These components may be executable, intermediate(e.g., IL) or interpreted(e.g., Java) code which communicate over a communication network(e.g., the Internet) using a communication protocol(e.g., TCP/IP).

Various embodiments may be programmed using an object-oriented programming language, such as SmallTalk, Java, C++, Ada, or C#(C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface(GUI) or perform other functions). Various aspects of the invention may be implemented as programmed or non-programmed elements, or any combination thereof.

Further, on each of the one or more computer systems that include one or more components of the system 100, each of the components may reside in one or more locations on the system. For example, different portions of the components of system 100 may reside in different areas of memory(e.g., RAM, ROM, disk, etc.) on one or more computer systems. Each of such one or more computer systems may include, among other components, a plurality of known components such as one or more processors, a memory system, a disk storage system, one or more network interfaces, and one or more busses or other internal communication links interconnecting the various components.

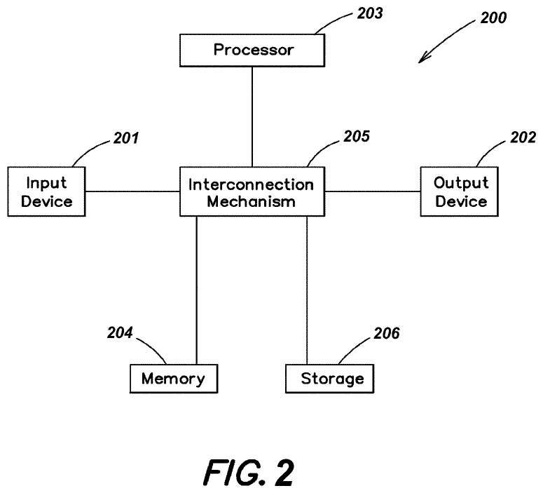

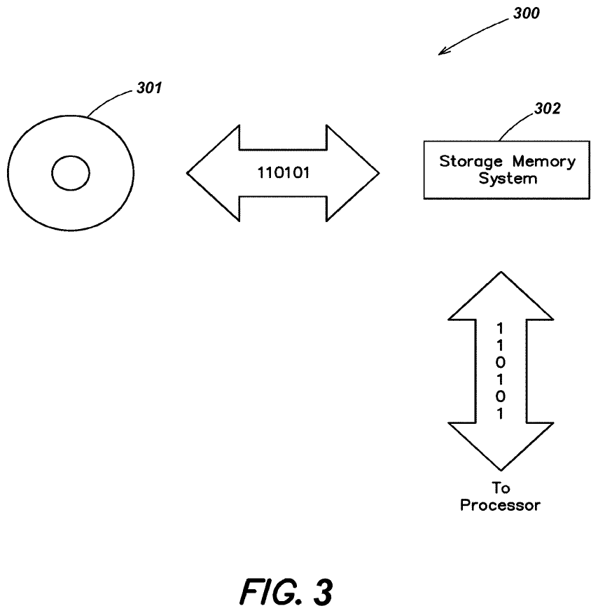

Any number of elements of the system 100 may be implemented and used to improve a computer system, for example, described in relation to FIGS. 2 and 3. In particular, FIG. 2 shows an example computer system 200 used to implement various aspects. FIG. 3 shows an example storage system that may be used. It should be appreciated that one or more of any type computer system may be used to partially or fully automate integration of the page fault handling system with the other systems and services according to various embodiments of the invention.

According to one embodiment, system 200 can be configured to generate and care management functions, secure and private date collection, remote monitoring, advise displays, remote chat sessions, compliance tracking and/or scoring, among other options. Further, the system may be located on a single computer or may be distributed among a plurality of computers attached by a communications network.

For example, various aspects of the invention may be implemented as specialized software executing in a general-purpose computer system 200(improving the system with new functionality and more efficient operations) such as that shown in FIG. 2. The computer system 200 may include a processor 203 connected to one or more memory devices 204, such as a disk drive, memory, or other device for storing data. Memory 204 is typically used for storing programs and data during operation of the computer system 200. Components of computer system 200 may be coupled by an interconnection mechanism 205, which may include one or more busses(e.g., between components that are integrated within a same machine) and/or a network(e.g., between components that reside on separate discrete machines). The interconnection mechanism 205 enables communications(e.g., data, instructions) to be exchanged between system components of system 200. Computer system 200 also includes one or more input/output devices 201-502, for example, a keyboard, mouse, trackball, microphone, touch screen, and one or more output devices 202, for example, a printing device, display screen, and/or speaker. In addition, computer system 200 may contain one or more interfaces(not shown) that connect computer system 200 to a communication network (in addition or as an alternative to the interconnection mechanism 205.

The storage system 206, shown in greater detail in FIG. 3, typically includes a computer readable and writeable nonvolatile recording medium 301 in which signals are stored that define a program to be executed by the processor or information stored on or in the medium 301 to be processed by the program. The medium may, for example, be a disk or flash memory. Typically, in operation, the processor causes data to be read from the nonvolatile recording medium 301 into another memory 302 that allows for faster access to the information by the processor than does the medium 301. This memory 302 is typically a volatile, random access memory such as a dynamic random access memory(DRAM) or static memory(SRAM). It may be located in storage system 206, as shown, or in memory system 204, not shown. The processor 203 generally manipulates the data within the integrated circuit memory 204 and 202 and then copies the data to the medium 301 after processing is completed. A variety of mechanisms are known for managing data movement between the medium 301 and the integrated circuit memory element 204 and 302, and the invention is not limited thereto. The invention is not limited to a particular memory system 204 or storage system 206.

The computer system may include specially-programmed, special-purpose hardware, for example, an application-specific integrated circuit (ASIC). Aspects of the invention may be implemented in software, hardware, or firmware, or any combination thereof. Further, such methods, acts, systems, system elements, and components thereof may be implemented as part of the computer system described above or as an independent component. Although computer system 200 is shown by way of example as one type of computer system upon which various aspects of the invention may be practiced, it should be appreciated that aspects of the invention are not limited to being implemented on the computer system as shown in FIG. 2. Various aspects of the invention may be practiced on one or more computers having a different architecture or components that that shown in FIG. 2.

Computer system 200 may be a general-purpose computer system that is programmable using a high-level computer programming language to implement the functions described herein, thereby providing new functionality and improved operation. Computer system 200 may also be implemented using specially programmed, special purpose hardware. In computer system 200, processor 203 is typically a commercially available processor such as the well-known Pentium, Core, Core Vpro, Xeon, or Itanium class processors available from the Intel Corporation. Many other processors are available. Such a processor usually executes an operating system which may be, for example, the Windows NT, Windows 2000 (Windows ME), Windows XP, Windows Vista, Windows 7 or 8 operating systems available from the Microsoft Corporation, MAC OS Snow Leopard, MAC OS Snow Lion operating systems available from Apple Computer, the Solaris Operating System available from Sun Microsystems, or UNIX available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server system that includes components distributed among one or more server systems that perform various functions according to various embodiments of the invention. These components may be executable, intermediate (e.g., IL), or interpreted(e.g., Java) code which communicate over a communication network (e.g., the Internet) using a communication protocol (e.g., TCP/IP). It should be appreciated that the invention is not limited to executing on any particular system or group of systems. Also, it should be appreciated that the invention is not limited to any particular distributed architecture, network, or communication protocol.

Having thus described several aspects and embodiments of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description is by way of example only.

Use of ordinal terms such as “first,” “second,” “ third,” “a,” “b,” “c,” etc., in the claims to modify or otherwise identify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A care management system comprising:
at least one processor operatively connected to a memory containing instructions, when executed cause the at least one processor to:
generate a user tailored care plan, comprising daily sequences of steps to accomplish the tailored care plan;
display customized interfaces to an end user specifying for a respective day each of the steps in the sequence associated with the respective day;
collect remote health information based on execution of the steps in the sequence; and
adjust organization of the sequence of steps based on analysis of the remote health information.

2. The system of claim 1, wherein the at least one processer is further configured to:
calculate and display a compliance score associated with performance of the steps in the sequence.

3. The system of claim 2, wherein the at least one processer is further configured to automatically trigger remote advice sessions in conjunction with an execution time for a respective step.

4. The system of claim 2, wherein the at least one processer is further configured to automatically trigger remote advice sessions in conjunction with an execution time for a respective step based on identifying a threshold compliance score.

5. The system of claim 1, wherein the system is further configured to display a customized interface for respective step execution, the customized interface displaying a single selection icon for presenting video instruction on step completion, and displaying a single selection icon for triggering a remote supervision session.

6. The system of claim 1, further comprising a knowledge base of education material on patient procedure, including self-care instruction.

7. (canceled)

8. The system of claim 7, wherein the at least one processor is further configured to filter material in the knowledge base according to a recovery phase associated with a respective user; and display of the customized interfaces includes display of an education display area, and wherein the at least one processor is configured to limit content displayed to the user within the education display area based, at least in part, on the recovery phase associated with the patient or a set of care plan task for a given day.

9-12. (canceled)

11. The system of claim 1, wherein the at least one processor is further configured to filter material in the knowledge base based, at least in part, on a recovery phase associated with a respective user and a set of care plan tasks for a given day.

12. The system of claim 9, wherein display of the customized interfaces includes display of an education display area, and wherein the at least one processor is configured to limit content display to the user within the education display area based, at least in part, on the recovery phase associated with the patient and the set of care plan tasks for a given day.

13. The system of claim 1, wherein display of the customized interfaces includes display of user entered goal information in conjunction with a summary display of a set of care plan tasks selected based on a target date.

14. The system of claim 1, wherein display of the customized interfaces includes display of a progress summary view, wherein the progress summary views is configured to access and display a care plan stage associated with a respective user, and display a set of prioritized task indicators.

15. The system of claim 1, wherein selection in the user interface of the prioritized task indicators is configured to transition the interface to a task view for completing an associated care plan task.

16. The system of claim 15, wherein at least some of the set of prioritized task indicators are associated with care plan tasks having designated times for completion, and wherein the interface is further configured to dynamically flash or reorder the display of at least one prioritized task indicator upon nearing the designated time for completion.

17. (canceled)

18. The system of claim 1, wherein the at least one processor is configured to capture care plan tasks and define progressive targets for display to respective users over time.

19. The system of claim 18, wherein the at least one processor is configured to adjust progressive targets and associated displays responsive to completion information captured or submitted by respective users.

20. The system of claim 19, wherein the at least one processor is configured to define progressive targets for blood sugar readings and adjust nutrition based activities responsive to individual patient progress.

21. The system of claim 1, wherein display of the customized interfaces includes display of an information aggregation display, wherein the at least one processor is configured to access information associated with scheduled task and capture needed pre-requisites for display to the user.

22. The system of claim 1, wherein display of the customized interfaces includes integration of acknowledgement indicators associated with pre-requisites.

23. (canceled)

24. The system of claim 1, wherein the at least one processor is configured to determine selection of procedure based modules associated with respective operations, activities, or procedures for patients.

25. The system of claim 24, wherein the system is configured to access recovery stage triggers associated with the procedure based modules to dynamically adapt information resources presented in the user interface to respective patients.

26-27. (canceled)

28. The system of claim 1, further comprising a communication module executed by the at least one processor, configured to present a communication channel connecting a patient and a care team as a group communication session.

29. The system of claim 28, wherein the at least one processor is configured to enforce communication according to group communication protocols.

30. (canceled)

31. The system of claim 30, wherein the at least one processor is configured to generate a symptom checker display configured to accept user reporting of any of the relevant symptoms.

32-64. (canceled)
